(19) United States
(12) Patent Application Publication
(10) Pub. No.: US 2013/0022973 A1
(43) Pub. Date: Jan. 24, 2013

(54) Multiplex Amplification for the Detection of Nucleic Acid Variations

(71) Applicants: Carl L. G. Hansen, Vancouver (CA); Oleh Petriv, Vancouver (CA); Kevin Heyries, Vancouver (CA); Kenneth J. Livak, San Jose, CA (US)

(72) Inventors: Carl L. G. Hansen, Vancouver (CA); Oleh Petriv, Vancouver (CA); Kevin Heyries, Vancouver (CA); Kenneth J. Livak, San Jose, CA (US)

(21) Appl. No.: 13/521,400

(22) PCT Filed: Jan. 14, 2011
(86) PCT No.: PCT/CA2011/000054

§ 371 Date: Sep. 26, 2012

Related U.S. Application Data
(60) Provisional application No. 61/282,299, filed on Jan. 15, 2010.
(60) Provisional application No. 61/282,298, filed on Jan. 15, 2010.

(51) Int. Cl. C12Q 1/68; G01N 21/64

(52) U.S. Cl. 435/6.11

(57) ABSTRACT

Kits, primers, and methods are provided herein for detecting relative target source to reference source ratios in a biological sample, by distributing the biological sample into discrete subsamples, wherein the biological sample includes, a plurality of target molecules on a target source; and a plurality of reference molecules on a reference source; providing target primers directed to one or more of the plurality of target molecules and reference primers directed to one or more of the plurality of reference molecules; performing digital amplification with the target primers and the reference primers; and detecting the presence or absence of amplified target products with target probes and detecting the presence or absence of amplified reference products with reference probes, wherein the ratio of amplified target products to amplified reference products is indicative of a relative amount of target source to reference source in a biological sample.

### CROSS REFERENCE TO RELATED APPLICATIONS

This application claims the benefit of U.S. Provisional Patent Application Ser. No. 61/282,298 entitled “MULTIPLEX AMPLIFICATION USING A COMMON PRIMER-DERIVED INTERNAL PROBE SEQUENCE” and Ser. No. 61/282,299 entitled “MULTIPLEX AMPLIFICATION USING A COMMON TEMPLATE-DERIVED INTERNAL PROBE SEQUENCE”, both filed on 15 Jan. 2010.

### FIELD OF THE INVENTION

The present invention relates to digital amplification methods. In particular, the invention relates to methods for detecting relative target source to reference source ratios in a biological sample.

### BACKGROUND

Chromosomal abnormalities and imbalances are responsible for a significant portion of genetic disorders in humans throughout their lives. Occasionally, during the processes of DNA replication, DNA repair, or recombination, errors occur in which the resulting cell comprises too many (or too few chromosomes), chromosomes with large deletions or duplications, etc. When such errors occur during meiosis, chromosomal abnormalities may cause serious birth defects. The occurrence after birth may also result in serious pathologies, including cancer.

Down's syndrome (DS), for example, is a genetic disorder affecting about 1 in 800 to 1 in 1000 newborns and is caused by the presence of 3 copies of chromosome 21, referred to as trisomy 21 (T21). The presence of a third chromosome results in over-expression of genes implicated in development, giving rise to phenotypical and cognitive abnormalities. In about 92% of the cases, DS is caused by a meiotic non-disjunction, whereby the resulting gamete has two copies of chromosome 21. In 3-4% of the cases, only a fraction of chromosome 21 translocates onto another chromosome prior to the first embryonic cell division and although there is no apparent trisomy karyotype, the T21 phenotype is present. Accordingly, the detection of fetal trisomy 21 (T21) is an important indication for prenatal diagnosis.

Down's syndrome may be determined early in the pregnancy (first trimester) although the standard prenatal screens are highly invasive and carry a finite risk of miscarriage (Thorsen et al., 2002). Maternal age plays an important role in the incidence of DS, increasing from 1 in 1,000 births for mothers younger than 30 to 1 in 12 births by age 49. In addition, women can be stratified according to their risk of carrying a fetus with T21 by several screening methods (such as ultrasonography and maternal serum biochemistry) but these techniques have limited sensitivity and high false positive rates. Due to the high risk of more sensitive invasive tests, only pregnant women above the age of 35, those with abnormal levels of serological markers (as determined by a triple screen measuring alpha-fetoprotein, human chorionic gonadotropin, and estriol), or those with a history of genetic disorders are tested by performing an amniocentesis. The amniocentesis procedure consists of inserting a needle into the uterus to collect a sample of amniotic fluid for karyotyping of fetal cells and carries significant risk of complications including infection, amniotic fluid leakage and, in 0.1% of the cases, miscarriage (Spencer, 2007). This is the only true diagnostic test since all existing non-invasive procedures are restricted to stratifying patients based on assessed risk. For instance, while up to 10% of women who undergo a triple screen will present with abnormal results, only 0.3% of these will have a fetus with a T21 genetic defect. This high false positive rate results in unnecessary anxiety, increased chance of complications and miscarriage (from unnecessary follow up testing), and increased cost of health care.

The use of DNA derived from various biological sources, including plasma or serum, for molecular genotyping and diagnosis has been in development for more than a decade (Boland 1996). Plasma and serum DNA from cancer patients has been shown to contain large quantities of tumor DNA (Chen et al. 1996; Nawroz et al. 1996; Anker et al. 1997). Lo et al. (1997) demonstrated that fetal DNA is present in maternal plasma and serum, and that detection of fetal DNA sequences was possible with just 10 ml of boiled plasma and serum. These observations suggested that plasma or serum DNA, or DNA from other biological samples including urine or feces, may be a useful source of material for the noninvasive and early diagnosis of various disorders associated with chromosomal abnormalities.

The use of cell-free fetal DNA in maternal plasma in noninvasive methods of prenatal diagnosis has been readily applied to sex-linked and certain single-gene disorders, but its use for fetal chromosomal aneuploidies has been a challenge (Costa et al. 2002; Lo, Hielm et al. 1998; Chiu et al. 2002; Lo and Chiu 2007). First, fetal nucleic acids coexist in maternal plasma with a high background of maternal nucleic acids that can often interfere with analysis (Lo, Tein et al. 1998). Second, fetal nucleic acids circulate in maternal plasma in a cell-free form, making it difficult to derive chromosome dosage information.

Several groups have been examining the feasibility of using digital PCR to detect fetal chromosomal abnormalities from maternal samples. One approach focuses on the detection of nucleic acid species that are fetal-specific, RNA molecules expressed by the placenta. Lo et al. (2007) demonstrated the use of digital PCR to determine the allelic imbalance of a single nucleotide polymorphism (SNP) on PLAC4 mRNA, a placenta-expressed transcript on chromosome 21, in the maternal plasma of women bearing trisomy 21 fetuses. Amplification of an equal number of each SNP allele was expected for a euploid fetus, whereas the amplification of a higher number of one allele is expected for an aneuploid fetus. Using this approach, Lo et al. were able to distinguish four aneuploid fetuses from nine normal healthy ones. Unfortunately, this approach is limited by its reliance on the heterozygosity of SNPs that lie on the chromosome of interest and are solely expressed by the placenta. The method is not applicable to a fetus homozygous for a single SNP allele. Moreover, the number of suitable mRNA SNPs that are sufficiently high expressed and informative is limited (Zimmerman et al. 2008).

Preferably, a noninvasive test for chromosomal abnormality detection is not dependent on the use of genetic polymorphisms. Lo et al. (2007) also used an assay comparing the dosage of a chromosome 21 sequence to a chromosome 1 sequence (Relative Chromosome Dosage; RCD) to demonstrate that digital PCR could be used to reliably detect fetal aneuploidy in artificial mixtures comprising as low as 25% fetal DNA. For a euploid fetus, the RCD of chromosome 12 and 21 sequences would be one. For a trisomy 21 fetus, there should be an overrepresentation amplified chromosome 21 sequences, and the degree of overrepresentation depends on the fetal DNA concentration in the sample. For example, when a sample comprises 50% fetal DNA, the theoretical RCD of chromosome 21 sequences to chromosome 12 sequences should be 1.25. However, this theoretical ratio decreases to 1.05 when analyzing a sample containing 10% fetal DNA. Using a similar assay to compare the dosage of a chromosome 21 sequence to a chromosome 12 sequence, Fan and Quake (2007) demonstrated that digital PCR could be used to discriminate between euploid and aneuploid samples. Unlike a SNP based approach, these methods do no depend on heterozygosity of a target locus, but rather rely on the relative dosage of target sequences on non-homologous chromosomes. Accordingly, these assays may be performed with no knowledge of the parents' or fetus's genotype.

Regardless, an insufficiently low fraction of fetal DNA in maternal samples remains the current barrier for using digital PCR for prenatal diagnosis of fetal aneuploidy. As reported by Lo et al., 7,680 molecules are required for correct disease classification 97% of the time with a fetal DNA concentration of 25%. To capture 7,680 molecules, Lo et al. estimate that 8 ml of maternal plasma would be required. This volume of plasma, obtainable from 15 ml of maternal blood, is at the limit of routine practice. Comparing analyses from several reports, Zimmerman et al. 2008 estimated that an approximately 20% fetal DNA concentration would be sufficient. On the other hand, fetal DNA present in maternal blood represents approximately 2 to 9.7% percent of the total DNA present in the cell-free serum during the first trimester (see, for e.g., Lo et al., 1997; Lo et al., 1998; Wachtel et al., 2001; Lee et al., 2002; Lun et al.; 2008) and 9.0% and 20.4% in the second and third trimesters, respectively (Lun et al.). Obviously, diagnosis of fetal chromosomal abnormalities is necessary before the third trimester, preferably within the first trimester when fetal-DNA concentrations are less than 10%.

Significant for early detection, decreasing concentration of fetal DNA, e.g., during early gestation, necessitates an increasingly larger sample for the RCD method. High confidence detection of a 1% enrichment of chromosome 21 is estimated to require sampling of approximately 500,000 molecules. This large number of DNA molecules raises a practical issue in the sample volume required for testing. It is estimated that during the first trimester of pregnancy the amount of fetal DNA circulating in the maternal serum is 5000 copies/mL (Lo et al., 1998; Li et al., 2004), so that a digital assay according to the method of Lo et al., or Fan and Quake, would require a total volume of approximately 100 mL of blood—an amount that is not practical for clinical screening. In addition, the purification and subsequent concentration of dilute genomic DNA to the required reaction volume would introduce significant losses, further aggravating this problem.

Several approaches have been investigated to overcome this obstacle including enrichment of fetal DNA by size selection (Li et al., 2004; Chinnapapagari et al., 2005), reduction of maternal background by fixation of hematopoietic cells in formaldehyde, and selective detection of epigenetic methylation markers specific to fetal development using bisulfite PCR (Lo et al., 2007; Lo and Chiu, 2008). Thus far these techniques have significant limitations due to increased labour requirements and inherent inefficiencies in sample preparation and processing. Dhallan et al. and Benachi et al. have reported the use of formaldehyde treatment to enrich fetal DNA concentrations to 25% and higher, although the effect of formaldehyde has not been universally observed by all groups (Chung et al. 2005; Chinnapapagari et al. 2005). While enrichment by size separation may also achieve these ratios, the amount of circulatory fetal DNA is limited. Moreover, size separation is generally a laborious method that leads to considerable loss of material, such that the gain in terms of the critical fraction of DNA achieved come at the expense of the critical amount of DNA necessary for the analysis. Accordingly, size separation would necessitate the efficient extraction of large and impractical volumes of blood from a subject.

Lo et al. 2007 and Zimmerman et al. 2008 have speculated that several sets of chromosome 21 and reference chromosome targets could be combined in an RCD approach to reduce the amount of maternal plasma necessary. However, the necessary multiplexed reactions are generally a major challenge, and are not possible for SNP-based approaches (Zimmerman et al. 2008). Multiplexing has its complications. Semi-quantitative methods using multiplex PCR have been described in the art, however these methods are not applicable to digital PCR. For instance, Casilli et al. (2002) teach a method for quantitative PCR of multiple short fluorescent fragments using multiplex PCR in which the amplified PCR fragments are be separated and individually quantified, e.g. by using electrophoretic or array based separation methods. In digital PCR, however, there is no downstream separation of PCR products—rather, the amplified products in each individual reaction are merely scored as a positive or negative, and it is the scoring of an extremely large number of individual reactions (each either positive or negative) that results in quantification.

Multiplex amplification also has inherent limitations, and generally requires significant optimization in order to achieve adequate results. Moreover, these limitations generally increase with the number of additional loci that are amplified or analyzed. For instance, each primer pair in the reaction should have relatively similar properties such as hybridization stringency, amplification efficiency, etc., in order to achieve optimal and consistent results. In addition to this, the inclusion of a plurality of probes into a single reaction can significantly increase the amount of non-specific primer hybridization and amplification, as well as the amount of primer-primer cross-reaction and cross-hybridization. When multiplexing in normal PCR, much of this non-specific “noise” can be removed by subsequent separation of the PCR products, for instance by gel electrophoresis or using microarrays. However, one of the advantages of digital amplification is rapid scoring of the amplification product, and this advantage would be lost if a downstream separation step were added. Therefore, issues of non-specific noise are highly relevant.

Consistent with the challenge that such multiplexing presents, recently disclosed digital amplification strategies to detect aneuploidies do not involve multiplexed probe-based detection of target molecules. Rather, US 20070202525 (Quake and Fan) discloses the use of a single target site on chromosome 21 and a single reference site on chromosome 12. US 20090029377 (Lo. et al.) rejects probe-based detection altogether and discloses the sequencing of digital amplification products to identify the chromosomal origin of the target molecule.

### SUMMARY

The present application is based, in part, on the discovery that the amplification from multiple genetic loci in a single digital amplification reaction is possible and may be accomplished by the use of sequence specific primers that result in the amplification of a common internal probe target sequence, thus allowing the detection of the amplified nucleic acids using a single probe type. In certain embodiments, the common internal probe target sequence may be a naturally occurring repetitive sequence on the original nucleic acid template which may be amplified by specifically sequence specific primers designed to flank a naturally occurring repetitive sequence. The use of a common internal probe target sequence in the amplified nucleic acids allows the use of a single probe type to detect the amplified nucleic acids, thus overcoming the inherent limitations of multiplexing in digital amplification, such as high background noise, high cross-reactivity between different probes, high levels of non-specific amplification, signal saturation and the like.

In accordance with one embodiment, there is provided a method of detecting relative target source to reference source ratios in a biological sample, the method including: (a) distributing the biological sample into discrete subsamples, wherein the biological sample includes, a plurality of target molecules corresponding to spaced apart target sites on a target source; and a plurality of reference molecules corresponding to spaced apart reference sites on a reference source; (b) providing a target primer subset of at least one digital amplification target primer pair directed to one or more of the plurality of target molecules and a reference primer subset of at least one digital amplification reference primer pair directed to one or more of the plurality of reference molecules; (c) performing digital amplification with the target primer subset and the reference primer subset; and (d) detecting the presence or absence of amplified target products with a subset of target probes and detecting the presence or absence of amplified reference products with a subset of reference probes, wherein the number of target probes and the number reference probes is less than the number of target and reference sites respectively, and wherein the ratio of amplified target products to amplified reference products is indicative of a relative amount of target source to reference source in a biological sample.

In accordance with another embodiment, there is provided a method of detecting a relative amount of target source to reference source in a biological sample, the method including: (a) distributing the biological sample into discrete subsamples, wherein the biological sample includes, a plurality of target molecules corresponding to spaced apart target sites on a target source; and a plurality of reference molecules corresponding to spaced apart reference sites on a reference source; (b) providing a target primer subset of at least one digital amplification target primer pair directed to one or more of the plurality of target molecules and a reference primer subset of at least one digital amplification reference primer pair directed to one or more of the plurality of reference molecules; (c) performing digital amplification with the target primer subset and the reference primer subset; and (d) detecting the presence or absence of amplified target products with a subset of target probes and detecting the presence or absence of amplified reference products with a subset of reference probes, wherein the target probes have a common template-derived internal probe target sequence and the reference probes have a common template-derived internal probe reference sequence, and wherein the ratio of amplified target products to amplified reference products is indicative of a relative amount of target source to reference source in a biological sample.

In accordance with another embodiment, there is provided a method of detecting the relative amount of target source to reference source in a biological sample, the method including: (a) providing a subset of target pre-amplification primer pairs, wherein each target pre-amplification primer pair is specific to at least one target site, and a subset of reference pre-amplification primer pairs, wherein each reference pre-amplification primer pair is specific to at least one reference site, and wherein each target pre-amplification primer pair has a first universal non-specific primer sequence and wherein each reference pre-amplification primer pair has a second universal non-specific primer sequence; (b) performing a pre-amplification reaction on the biological sample with the first and second subsets to synthesize the target molecules and the reference molecules, wherein the biological sample includes additional target and reference molecules; (c) distributing the biological sample into discrete subsamples, wherein the biological sample includes a plurality of target molecules corresponding to spaced apart target sites on a target source; and a plurality of reference molecules corresponding to spaced apart reference sites on a reference source; providing a target primer subset of at least one digital amplification target primer pair directed to one or more of the plurality of target molecules and a reference primer subset of at least one digital amplification reference primer pair directed to one or more of the plurality of reference molecules; performing digital amplification with the target primer subset and the reference primer subset; and (f) detecting the presence or absence of amplified target products with a subset of target probes and detecting the presence or absence of amplified reference products with a subset of reference probes, wherein the number of target probes and the number reference probes is less than the number of target and reference sites respectively, and wherein the ratio of amplified target products to amplified reference products is indicative of a relative amount of target source to reference source in a biological sample.

The target probes may be designed to identify a common template-derived internal probe target sequence and the reference probes may be designed to identify a common template-derived internal probe reference sequence. Alternatively, the target primer subset primer pairs may be combination probe and primer molecules and the reference primer subset primer pairs may be combination probe and primer molecules. (for example, Scorpion™)

Alternatively, the method may further include a pre-amplification prior to distributing the biological sample into discrete subsamples, wherein the pre-amplification includes: (a) providing a subset of target pre-amplification primer pairs, wherein each target pre-amplification primer pair is specific to at least one target site, and a subset of reference pre-amplification primer pairs, wherein each reference pre-amplification primer pair is specific to at least one reference site, and wherein each target pre-amplification primer pair has a first universal non-specific primer sequence and wherein each reference pre-amplification primer pair has a second universal non-specific primer sequence; and (b) performing a pre-amplification reaction on the biological sample with the first and second subsets to synthesize the target molecules and the reference molecules.

The ratio of amplified target products to amplified reference products may be indicative of the presence or absence of a chromosomal abnormality. The target source may be all or part of a chromosome and the reference source may be all or part of a different chromosome.

The target source may be selected from human chromosomes: X; Y; 8; 9; 12; 13; 16; 18; 21; and 22. The target source may be selected from one or more of human chromosomes: X; Y; 8; 9; 12; 13; 16; 18; 21; and 22. The reference source may be selected from one or more of human chromosomes: 1; 2; 3; 4; 5; 6; 7; 10; 11; 14; 15; 17; 19; and 20. The reference source may be selected from human chromosomes: 1; 2; 3; 4; 5; 6; 7; 10; 11; 14; 15; 17; 19; and 20.

The target primer subset of primers may be added to a first subset of discrete subsamples and the reference primer subset of primers may be added to a second subset of discrete subsamples. The discrete subsamples may include on average about 1.59 target molecules or reference molecules. The discrete subsamples may have between 0-3 target or reference molecules. The discrete subsamples may have on average between 0-3 target or reference molecules. Alternatively, the discrete subsamples may have on average between 0-2.5 target or reference molecules. Alternatively, the discrete subsamples may have on average between 0-2.0 target or reference molecules. Alternatively, the discrete subsamples may have on average between 0-1.5 target or reference molecules. Alternatively, the discrete subsamples may have on average about 1 target or reference molecule.

The target primer subset may be directed to at least 2 target sites and the reference primer subset is directed to at least 2 reference sites. Alternatively, the target primer subset may be directed to at least 3 target sites and the reference primer subset is directed to at least 3 reference sites. Alternatively, the target primer subset may be directed to at least 4 target sites and the reference primer subset is directed to at least 4 reference sites. Alternatively, the target primer subset may be directed to at least 5 target sites and the reference primer subset is directed to at least 5 reference sites. Alternatively, the target primer subset may be directed to at least 6 target sites and the reference primer subset is directed to at least 6 reference sites. Alternatively, the target primer subset may be directed to at least 7 target sites and the reference primer subset is directed to at least 7 reference sites. Alternatively, the target primer subset may be directed to at least 8 target sites and the reference primer subset is directed to at least 8 reference sites. Alternatively, the target primer subset may be directed to at least 9 target sites and the reference primer subset is directed to at least 9 reference sites. Alternatively, the target primer subset may be directed to at least 10 target sites and the reference primer subset is directed to at least 10 reference sites. Alternatively, the target primer subset may be directed to at least 11 target sites and the reference primer subset is directed to at least 11 reference sites. Alternatively, the target primer subset may be directed to at least 12 target sites and the reference primer subset is directed to at least 12 reference sites. Alternatively, the target primer subset may be directed to at least 13 target sites and the reference primer subset is directed to at least 13 reference sites. Alternatively, the target primer subset may be directed to 2-20 target sites and the reference primer subset is directed to 2-20 reference sites.

Each site on the target source is separated by at least 10 kb and each site on the reference source is separated by at least 10 kb. Alternatively, each site on the target source is separated by at least 1 kb and each site on the reference source is separated by at least 1 kb. Alternatively, each site on the target source is separated by at least 2 kb and each site on the reference source is separated by at least 2 kb. Alternatively, each site on the target source is separated by at least 3 kb and each site on the reference source is separated by at least 3 kb. Alternatively, each site on the target source is separated by at least 4 kb and each site on the reference source is separated by at least 4 kb. Alternatively, each site on the target source is separated by at least 5 kb and each site on the reference source is separated by at least 5 kb. Alternatively, each site on the target source is separated by at least 6 kb and each site on the reference source is separated by at least 6 kb. Alternatively, each site on the target source is separated by at least 7 kb and each site on the reference source is separated by at least 7 kb. Alternatively, each site on the target source is separated by at least 8 kb and each site on the reference source is separated by at least 8 kb. Alternatively, each site on the target source is separated by at least 9 kb and each site on the reference source is separated by at least 9 kb. Alternatively, each site on the target source is separated by at least 11 kb and each site on the reference source is separated by at least 11 kb. Alternatively, each site on the target source is separated by at least 12 kb and each site on the reference source is separated by at least 12 kb. Alternatively, each site on the target source is separated by at least 13 kb and each site on the reference source is separated by at least 13 kb. Alternatively, each site on the target source is separated by at least between 1 kb-20 kb and each site on the reference source is separated by at least between 1 kb-20 kb.

The biological sample may be blood, blood plasma, blood serum, urine, feces, saliva, or transcervical lavage. The biological sample may be maternal blood serum.

The ratio of amplified target products to amplified reference products may be indicative of the presence or absence of fetal aneuploidy. The target source may be human chromosome 21. The ratio of amplified target products to amplified reference products may be indicative of the presence or absence of trisomy 21 in the fetal chromosomes.

The at least one digital amplification target primer pair may be selected from one or more of the following primer pairs:
(a) SEQ ID NO: 1 and SEQ ID NO: 2;
(b) SEQ ID NO: 3 and SEQ ID NO: 4;
(c) SEQ ID NO: 5 and SEQ ID NO: 6;
(d) SEQ ID NO: 7 and SEQ ID NO: 8;
(e) SEQ ID NO: 9 and SEQ ID NO: 10;
(f) SEQ ID NO: 11 and SEQ ID NO: 12;
(g) SEQ ID NO: 13 and SEQ ID NO: 14; and
(h) SEQ ID NO: 15 and SEQ ID NO: 16.

The universal primer pair may be SEQ ID NO: 17 and SEQ ID NO: 18.

The template derived internal probe sequence may be SEQ ID NO: 19. The labeled template derived internal probe sequence may be SEQ ID NO: 20. Alternative labeling methodologies are described herein and are known to persons of skill in the art. Furthermore, alternative primer pairs design strategies are described herein and are known to persons of skill in the art.

In accordance with another embodiment, there are provided digital amplification target primer pairs for human chromosome 21, which may be selected from one or more of the following primer pairs: (a) SEQ ID NO: 1 and SEQ ID NO: 2; (b) SEQ ID NO: 3 and SEQ ID NO: 4; (c) SEQ ID NO: 5 and SEQ ID NO: 6; (d) SEQ ID NO: 7 and SEQ ID NO: 8; (e) SEQ ID NO: 9 and SEQ ID NO: 10; (f) SEQ ID NO: 11 and SEQ ID NO: 12; (g) SEQ ID NO: 13 and SEQ ID NO: 14; and (h) SEQ ID NO: 15 and SEQ ID NO: 16.

In accordance with another embodiment, there is provided a template derived internal probe sequence (SEQ ID NO: 19) and a labeled template derived internal probe sequence (SEQ ID NO: 20).

In accordance with another embodiment, there is provided a kit for detecting the relative amount of target source to reference source in a biological sample, the kit including: (a) a target primer subset of at least 8 digital amplification target primer pairs directed to a plurality of target molecules and a reference primer subset of at least 8 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 2 digital amplification target primer pairs directed to a plurality of target molecules and a reference primer subset of at least 2 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 3 digital amplification target primer pairs directed to a plurality of target molecules and a reference primer subset of at least 3 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 4 digital amplification target primer pairs directed to a plurality of target molecules and a reference primer subset of at least 4 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 5 digital amplification target primer pairs directed to a plurality of target molecules and a reference primer subset of at least 5 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 6 digital amplification target primer pairs directed to a plurality of target molecules and a reference primer subset of at least 6 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 7 digital amplification target primer pairs directed to a plurality of target molecules and a reference primer subset of at least 7 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 9 digital amplification target primer pairs directed to a plurality of target molecules and a reference primer subset of at least 9 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

In accordance with another embodiment, there is provided a kit for detecting the relative amount of target source to reference source in a biological sample, the kit including: (a) a target primer subset of at least 10 digital amplification target primer pairs directed to a plurality of target molecules and a reference primer subset of at least 10 digital amplification reference primer pairs directed to a plurality of reference molecules, wherein the target molecules may be situated on a human chromosome selected from the following: X; Y; 8; 9; 12; 13; 16; 18; 21; and 22 and wherein the reference molecules may be situated on a human chromosome selected from the following: 1; 2; 3; 4; 5; 6; 7; 10; 11; 14; 15; 17; 19; and 20; (b) an amplification buffer for digital amplification using the target primer pairs and the reference primer pairs; and (c) a target probe specific for the target primer pair amplification products and a reference probe specific for the reference primer pair amplification products.

The at least one digital amplification target primer pair may be selected from one or more of the following primer pairs: (a) SEQ ID NO: 1 and SEQ ID NO: 2; (b) SEQ ID NO: 3 and SEQ ID NO: 4; (c) SEQ ID NO: 5 and SEQ ID NO: 6; (d) SEQ ID NO: 7 and SEQ ID NO: 8; (e) SEQ ID NO: 9 and SEQ ID NO: 10; (f) SEQ ID NO: 11 and SEQ ID NO: 12; (g) SEQ ID NO: 13 and SEQ ID NO: 14; and (h) SEQ ID NO: 15 and SEQ ID NO: 16.

### BRIEF DESCRIPTION OF THE DRAWINGS

In drawings which illustrate embodiments of the invention:

FIG. 1 shows the scaling of digital PCR precision with assay number and template concentration, where the total number of chambers is N and the expected number of molecules per chamber is λ. Precision is plotted for N=1,000, 10,000, 100,000, and 1,000,000 across a range of λ from 0.01 to 5.

FIG. 2 is a numerical calculation of the separation in the measured mean of two alleles varying by 1% using digital PCR as a function of the number of chambers.

FIG. 3 is a digital PCR analysis of two-step amplification protocol using primers with gene specific regions and flanking universal primer sequences. (A) schematic diagram for a potential multiplex digital PCR, which includes a pre-amplification step. (B) Digital micrographs of the 40th PCR cycle from multiplex digital PCR with progressively higher numbers of molecules detected corresponding to increasing the number of gene-specific primer pairs (see TABLE 3 herein) used in the pre-amplification protocol at constant template concentration.

### DETAILED DESCRIPTION

Primer pairs as described herein may be designed such that the nucleic acid amplification product of each of the primer pairs comprises a common internal probe target sequence. The common internal probe target sequence may be a naturally occurring repetitive nucleic acid sequence found on the original target source or reference source nucleic acid template. The amplification primers may be chosen from the primers listed in TABLE 3 (SEQ ID NOs: 1-16). Alternatively, a person of skill in the art would be able to design primer pairs based on the desired target source or reference source using methods known to persons skilled in the art. For example, the design of primers may take into consideration the restriction sites in a source of interest. Further, the design of suitable primers may take into consideration the presence of repeat sequences within the chromosomal areas of interest. Suitable primers may include, without limiting the foregoing, sequences that would amplify regions wherein the chromosomal distance from a first repeat sequence to a second repeat sequence is greater or equal to the chromosomal distance from the corresponding restriction sites. Alternatively, the design of primers may include the gene specific sequences of interest. Alternatively, the design of primers may include both the gene specific sequences of interest together with universal primer sequences (for example, TABLE 3). Alternatively, the design of primers may include gene specific sequences of interest together with universal primer sequences along with a probe sequence as described herein. Digital amplification may occur via digital PCR (dPCR) or rolling circle amplification.

Other amplification methods are known to those skilled in the art. For example, amplification techniques, without limitation, may include BEAMing™, Droplet Digital™ PCR (QuantaLife™), and commercially available products through Life Technologies™, and Raindance Technologies™. For example, BEAMing™ is generally understood to include four steps: pre-amplification, emulsion PCR, hybridization, and flow cytometry (see, for example, Dressman at al. (2006), PNAS 100: 8817-22; Diehl at al. (2005), PNAS 102:16368-73; Diehl et al. (2006) Nat. Method. 3(7):551-9; and Li et al. (2006) Nat. Method. 3(2): 95-7). Alternatively, for example, Droplet Digital™ PCR (QuantaLife™) involves the following general methods: (i) making the droplets; (ii) amplifying the samples (either DNA/RNA) using a standard thermal cycler; and (iii) reading the reaction by way of a fluorescence detector.

The detection methods described herein and known in the art may be suitable for identifying a fetal chromosomal abnormality in a maternal biological sample. A method may include the following steps: 1) Obtaining a maternal biological sample, which includes maternal and fetal nucleic acids (for example, maternal blood or maternal blood serum); 2) Performing a first multiplex digital amplification reaction on the maternal biological sample, wherein the first multiplex digital amplification reaction includes i) a plurality of primer pairs specific to a target chromosome, wherein the primer pairs are designed such that the nucleic acid amplification product of each of the primer pairs includes a common internal probe target sequence; and ii) a hybridization probe which binds to the common internal probe target sequence; 3) Performing a second multiplex digital amplification reaction on the maternal biological sample, wherein the second multiplex digital amplification reaction includes i) a plurality of primer pairs specific to the non-target (reference) chromosome, wherein the primer pairs are designed such that the nucleic acid amplification product of each of the primer pairs includes a common internal probe target sequence; and ii) a hybridization probe which binds to the common internal probe target sequence; and 4) Measuring the relative amount of target and non-target nucleic acid sequences amplified, wherein the relative amount amplified is indicative of the presence of a fetal chromosomal abnormality for the target chromosome.

An alternative method of identifying a fetal chromosomal abnormality in a maternal biological sample from a subject, may include the following steps: 1) Obtaining a maternal biological sample including maternal and fetal nucleic acids; 2) Performing a pre-amplification reaction on the maternal biological sample, wherein the reaction includes: i) a plurality of primer pairs specific to the target chromosome, wherein the primer pairs are designed such that the nucleic acid amplification product of each of the primer pairs includes a common internal probe target sequence, and wherein each primer pair also includes a universal priming site on each primer of the pair; and ii) a plurality of primer pairs specific to the non-target chromosome, wherein the primer pairs are designed such that the nucleic acid amplification product of each of the primer pairs includes a common internal probe target sequence, and wherein each primer pair also includes a universal priming site on each primer of the pair; 3) Performing a first multiplex digital amplification reaction on the pre-amplification reaction product, wherein the first multiplex digital amplification reaction includes i) one or more universal primers designed to hybridize to the universal primer binding sites incorporated into the pre-amplification primers; and ii) a hybridization probe which binds to the common internal probe target sequence from the target chromosome amplified products; 4) Performing a second multiplex digital amplification on the maternal biological sample, wherein the second multiplex digital amplification includes i) one or more universal primers designed to hybridize to the universal primer binding sites incorporated into the pre-amplification primers; and ii) a hybridization probe which binds to common internal probe sequence from the non-target chromosome amplified products; and 5) Measuring the relative amount of target and non-target sequences amplified, wherein the relative amount amplified is indicative of the presence of a fetal chromosomal abnormality for the target chromosome.

An alternative method of identifying a fetal chromosomal abnormality in a maternal biological sample from a subject is described herein. The method may include the following steps: 1) Obtaining a maternal biological sample including maternal and fetal nucleic acids; 2) Performing a first multiplex digital amplification reaction on the maternal biological sample, wherein the first multiplex digital amplification reaction includes i) a plurality of primer pairs specific to the target chromosome, wherein the primer pairs may be designed such that the nucleic acid amplification product of each of the primer pairs includes a common internal probe target sequence; and ii) a hybridization probe which binds to the common internal probe target sequence; 3) Performing a second multiplex digital amplification reaction on the maternal biological sample, wherein the second multiplex digital amplification reaction includes i) a plurality of primer pairs specific to the non-target chromosome, wherein the primer pairs are designed such that the nucleic acid amplification product of each of the primer pairs includes a common internal probe target sequence; and ii) a hybridization probe which binds to the common internal probe target sequence; and 4) Measuring the relative amount of target and non-target nucleic acid sequences amplified, wherein the relative amount amplified is indicative of the presence of a fetal chromosomal abnormality for the target chromosome.

Alternatively, a method of identifying a fetal chromosomal abnormality in a maternal biological sample from a subject may include the following steps: 1) Obtaining a maternal biological sample comprising maternal and fetal nucleic acids; 2) Performing a pre-amplification reaction on the maternal biological sample, wherein the reaction includes: i) a plurality of primer pairs specific to the target chromosome, wherein the primer pairs may be designed such that the nucleic acid amplification product of each of the primer pairs includes a common internal probe target sequence, and wherein each primer pair also may include a universal priming site on each primer of the pair; and ii) a plurality of primer pairs specific to the non-target chromosome, wherein the primer pairs may be designed such that the nucleic acid amplification product of each of the primer pairs includes a common internal probe target sequence, and wherein each primer pair also comprises a universal priming site on each primer of the pair; 3) Performing a first multiplex digital amplification reaction on the pre-amplification reaction product, wherein the first multiplex digital amplification reaction includes i) one or more universal primers designed to hybridize to the universal primer binding sites incorporated into the pre-amplification primers; and ii) a hybridization probe which binds to the common internal probe target sequence from the target chromosome amplified products; 4) Performing a second multiplex digital amplification on the maternal biological sample, wherein the second multiplex digital amplification includes i) one or more universal primers designed to hybridize to the universal primer binding sites incorporated into the pre-amplification primers; and ii) a hybridization probe which binds to common internal probe sequence from the non-target chromosome amplified products; and 5) Measuring the relative amount of target and non-target sequences amplified, wherein the relative amount amplified is indicative of the presence of a fetal chromosomal abnormality for the target chromosome.

Amplification primers, and methods of their use are described herein (see TABLE 3), may be designed to amplify chromosome-specific short repetitive elements that are dispersed across a chromosome of interest, for example chromosome 21. Primers may also be designed as pairs of primers that each flank a different short repetitive element on the given chromosome, such that each primer pair will amplify a nucleic acid sequence that contains the short repetitive element. A set of such primer pairs thus provides the advantage of amplifying a series of nucleic acid fragments from a single chromosome of interest, for instance chromosome 21, which all contain a common nucleic acid sequence. The common nucleic acid element may thus be utilized, for instance, for detection of the amplified nucleic acid fragments. In certain embodiments, a hybridization probe may be designed which specifically binds to the short repetitive element. The hybridization probe may be, without limiting the foregoing, an LNA FRET probe.

The primers in TABLE 3 may be useful as amplification primers, in a digital amplification (for example dPCR or rolling circle amplification) and may be incorporated in pairs into a molecular inversion probe or primer for a rolling circle amplification. The primers may be used to amplify specific chromosomal regions of the genome, and a plurality of primer pairs may be used for multiplexing an amplification reaction. The primers may find further use as corresponding pairs of amplification primers for the quantification of nucleic acids, for instance for the quantification of fetal and/or maternal chromosomal nucleic acids for the identification or detection of fetal aneuploidy, using digital amplification.

The primers described herein may be utilized for multiplex amplification reactions, for instance when performing digital amplification. Since the primer sets can amplify specific nucleic acid sequences which contain a common internal nucleic acid element. Accordingly, it may be possible to utilize a single probe for the detection of each of the amplified sequences, which may overcome certain disadvantages of using multiplexing in digital amplification reactions. For example, in digital PCR, many of the limitations of multiplexing reactions have precluded or undermined the use of multiplexing in dPCR.

A method of multiplexing in a digital amplification reaction is described herein, wherein the method may include performing a digital amplification reaction which comprises a plurality of primer pairs, wherein the primer pairs may be designed such that the nucleic acid amplification product of each of the primer pairs includes a common internal probe target sequence. The common internal probe target sequence may be a naturally occurring repetitive nucleic acid sequence found on the original nucleic acid template. The amplification primers may be chosen from the primers listed in TABLE 3 (SEQ ID NOs: 1-16). The digital amplification reaction may be, but is not limited to, PCR or rolling circle amplification.

Hybridization probe(s) may further comprise a tag, for instance a fluorescent tag. The hybridization probe may be a FRET probe or an LNA FRET probe. Alternatively, the first and second multiplex amplification reactions may be performed in a single reaction. The common internal probe target sequence from the first and second multiplex reactions, and thus also the hybridization probes designed to bind to these sequences, may be different from each other. Hybridization probes from the first and second multiplex reactions may be tagged with different tags, for instance different colored fluorescent tags. Alternatively, the maternal biological sample may be prepared prior to performing the multiplex amplification reaction, for instance to purify the nucleic acids, or to remove elements of the sample that may interfere with the multiplex reaction. The common internal probe target sequence may be a naturally occurring repetitive nucleic acid sequence found on the original nucleic acid template. The amplification primers may be chosen from the primers listed in TABLE 3 (SEQ ID NOS: 1-16).

As used herein, the term “subject” refers to an animal, such as a bird or a mammal. A specific animal may include rat, mouse, dog, cat, cow, sheep, camel, horse, goat, pig, chicken, duck, or primate. A subject may alternatively be a human, and be alternatively referred to as a patient. A subject may be a person at risk of developing a pathology. A subject may further be a pregnant female. A subject may further be a transgenic animal. A subject may further be a rodent, such as a mouse or a rat.

As used herein, the term “biological sample” refers to any sample that is taken from a subject (e.g., a human), and contains one or more nucleic acid molecule(s) of interest. In various embodiments, the biological sample may be blood, blood plasma, blood serum, urine, feces, saliva, or transcervical lavage. The biological sample may be a maternal sample from a pregnant female subject, such as urine, plasma, serum, that comprises a mixture of maternal and fetal DNA. Alternatively, the biological sample may be from a subject with a neoplasm, or at risk of developing a neoplasm correlated with a chromosomal abnormality. In another aspect, the biological sample comprises amplified copies of one or more nucleic acids of interest that have been taken from a subject.

As used herein, the term “chromosomal region” refers to a chromosome, or portion thereof, of sufficient size or significance that changes in the dosage of this region, through deletions, duplications, or non-disjunctions, may result in a phenotype clinically relevant condition.

As used herein, the term “chromosomal abnormality” may be defined as an abnormal karyotype, which includes abnormalities in the number (i.e., aneuploidy), or of composition of the individual chromosomes (for example, additions, amplifications, deletions, duplications, rearrangements, translocations, or tranversions).

As used herein, the term “aneuploidy” may be defined as any variation in chromosome number that involves individual chromosomes rather than entire sets of chromosomes. There may be fewer chromosomes (i.e., monosomy), as in Turner's syndrome in which a female has only one X chromosome, or more chromosomes (i.e., trisomy), as in Down's syndrome where there are three copies of chromosome 21. Aneuploidy is known to occur in relation to X, Y, 8, 9, 12, 13, 16, 18, 21, and 22 chromosomes and may be partial, full, or mosaic.

As used herein, the term “chromosomal imbalance” refers to a situation wherein the dosage of a chromosomal region of interest in a subject differs from that of a reference subject. Alternatively, chromosomal imbalance may refer to a condition wherein the dosage of a chromosomal region in a cell of a subject differs from a reference cell of the same subject. A chromosomal imbalance may further include the presence of an X and Y chromosome, such as in the genome of a male fetus. A chromosomal imbalance may further include a chromosomal abnormality as defined above. It could further include one or more allelic differences between the same chromosomal region of a homologous chromosome pair.

Detecting relative target source to reference source ratios in a biological sample may be useful in identifying chromosomal abnormalities or chromosomal imbalance. For example, aneuploidy or neoplasms.

As used herein, the term “target source” refers to a chromosomal region for which variations in dosage may be possible within a biological sample of interest. The chromosomal region may be a clinically-relevant chromosomal region that is associated with a prevalence for chromosomal imbalance or chromosomal abnormality. Alternatively, the chromosomal region may be a segment of a chromosome within which the frequency of recombination is significantly less than 50%, such that the segment is generally inherited as a single unit. Alternatively, the chromosomal region may consist of, be a portion of, an allosome. For example, in humans a target source may be a chromosome know to undergo aneuploidy (for example, X, Y, 8, 9, 12, 13, 16, 18, 21, and 22).

As used herein, the term “reference source” refers to a chromosomal region for which variations in dosage are not expected within a biological sample of interest (background regions). The chromosomal region may be a non-clinically-relevant chromosomal region that is not associated with a prevalence for chromosomal imbalance or chromosomal abnormality. The chromosomal region will generally consist of an autosomal chromosome or portion thereof. For example, in humans a reference source may be a chromosome know to be euploid (for example, 1; 2; 3; 4; 5; 6; 7; 10; 11; 14; 15; 17; 19; and 20).

As used herein, the term “relative target source to reference source ratio” refers to the relative number of a target source to a reference source in a biological sample. This ratio may be used to determine a number of characteristics of a fetus, including, but not limited to the presence of aneuploidy, deletions, amplifications, mosaics, or the sex of twins, or identify the presence of a neoplastic condition in a subject. This ratio may further be used to detect the presence of cells in the body comprising a chromosomal abnormality.

As used herein, the term “target site” refers to a discrete, identifiable region of a target source. Each target site will generally be spaced apart from an adjacent target site by a sequence longer than the average DNA fragment size in the biological sample. In some embodiments, the biological sample may be treated with a restriction enzyme prior to distribution into discrete subsamples for the digital amplification reaction, wherein the average fragment size generally reflects the frequency with which the restriction enzyme cleaves the DNA. In one aspect of the invention, the average fragment size will be between about 300 nucleotides and 1000 nucleotides in length. In one aspect, each target site is separated from an adjacent target site by about 10 kb. In another aspect, the two furthest separated target sites will be sufficiently close such that the frequency of recombination between them is significantly less than 50%.

As used herein, the term “reference site” refers to a discrete, identifiable region of a reference source. Each reference site will generally be spaced apart from an adjacent reference site by a sequence longer than the average fragment size in the sample. In some embodiments, the biological sample may be treated with a restriction enzyme prior to distribution into discrete subsamples for the digital amplification reaction, wherein the average fragment size generally reflects the frequency with which the restriction enzyme cleaves the DNA. In one aspect, each reference site is separated from an adjacent target reference site by about 10 kb. In another aspect, the two furthest separated reference sites will be sufficiently close such that the frequency of recombination between them is significantly less than 50%.

As used herein, the term “target molecule” refers to a DNA molecule within a biological sample that comprises a sequence corresponding to target site of a target source. A target molecule will generally comprise a sequence corresponding to a single target site. Target molecules generally comprise fragments of chromosomes, and may be double stranded or single stranded. In one aspect, a target molecule may be an amplified copy of a DNA molecule, e.g. produced during a pre-amplification reaction, within a biological sample. Where the target molecule is such an amplified copy, the target molecule may comprise a universal non-specific primer sequence that is introduced through a pre-amplification reaction. Where the target molecule is an amplified copy, the target molecule may include a probe target sequence introduced through a pre-amplification reaction.

As used herein, the term “reference molecule” refers to a DNA molecule within a biological sample that comprises a sequence corresponding to reference site of a reference source. A reference molecule will generally comprise a sequence corresponding to a single reference site. Reference molecules generally comprise fragments of chromosomes, and may be double stranded or single stranded. In one aspect, a reference molecule may be an amplified copy of a DNA molecule, e.g., produced during a pre-amplification reaction, within a biological sample. Where the reference molecule is an amplified copy, the reference molecule may comprise a universal non-specific primer sequence that is introduced through a pre-amplification reaction. Where the reference molecule is an amplified copy, the target may include a probe reference sequence introduced through a pre-amplification reaction. Where the reference molecule is an amplified copy, the reference molecule may include a probe reference sequence introduced through a pre-amplification reaction.

As used herein, the term “target primer” refers to an oligonucleotide primer which can be used to prime the amplification of a target molecule. A target primer may be specific to a single target site. Alternatively, a target primer may be common to at least two target sites. A target primer may be common to all target sites (i.e., a universal target primer). The sequence to which a target primer anneals may be introduced to a target molecule during a pre-amplification step with pre-amplification primers (e.g., as for the addition of a universal non-specific primer sequence).

As used herein, the term “subset of target primers” refers to a set of target primers. In one aspect, the subset of target primers includes a single pair of target primers that is common to all target molecules. In another aspect, the subset includes a number of target primer pairs that may be equal to the number of target sites.

As used herein, the term “reference primer” refers to an oligonucleotide primer which can be used to prime the amplification of a reference molecule. In one aspect, a reference primer may be specific to a single reference site. In another aspect, a reference primer may be common to at least two reference sites. In another aspect, the reference primer is common to all reference sites (i.e., a universal reference primer). In one aspect, the sequence to which the reference primer anneals may be introduced to the reference molecule during a pre-amplification step with pre-amplification primers (i.e., as with the addition of a universal non-specific primer sequence).

As used herein, the term “subset of reference primers” refers to a set of reference primers. In one aspect, the subset of reference primers includes a single pair of reference primers that is common to all reference molecules. In another aspect, the subset includes a number of reference primer pairs that may be equal to the number of reference sites.

As used herein, the term “universal non-specific primer sequence” refers to the sequence of a target primer or reference primer that may be used to amplify all target or reference molecules, respectively. Accordingly, a “universal target primer” or “universal reference primer” as used herein refers to a target primer or reference primer that may be used to amplify all target or reference molecules, respectively. An example of universal primers are as follows: GTTGTAAAACGACGGCCAGT (universal forward primer, SEQ ID NO: 17) and CACAGGAAACAGCTATGACC (universal reverse primer, SEQ ID NO: 18).

As used herein, the term “universal priming site” refers to a nucleic acid sequence on a target molecule which is complementary to a universal non-specific primer sequence. A universal priming site may be introduced to a target or reference molecule during a pre-amplification step using pre-amplification primers. The inclusion of a universal non-specific primer sequence into each pre-amplification primer of a given pre-amplification primer pair during a pre-amplification reaction will thus lead to the production of target and reference molecules which contain a universal priming site at each end. In certain embodiments, there are thus provided target and reference pre-amplification primer pairs which comprise universal priming sites, and methods of their use. An example of a universal priming site would be the reverse complement of SEQ ID NO: 17 and SEQ ID: NO 18 detailed herein.

As used herein, the term “PCR, which is also known as “polymerase chain reaction”, refers to a process of nucleic acid amplification, wherein a pair of PCR primers are introduced into a reaction mixture comprising a target nucleic acid sequence, a polymerase such as DNA polymerase or thermostable DNA polymerase, and other necessary components to achieve amplification, such as nucleotides (usually deoxynucleoside triphosphates), buffer and the like. Accurate iterative thermocycling—successive heating and cooling—of the reaction allows the primers to anneal to the complementary target sequence, followed by extension of the primer along the nucleic acid template, followed by denaturation of the newly formed nucleic acid product from the template, such newly formed nucleic acids thus being available to act as templates in subsequent cycles of the reaction. Methods of PCR primer design and PCR implementation, including variations thereof, are well known in the art (see, for e.g., Sambrook, Joseph. and Russell, David W. and Cold Spring Harbor Laboratory. Molecular cloning: a laboratory manual/Joseph Sambrook, David W. Russell 2001).

As used herein, the term “digital amplification” describes a method by which a biological sample is distributed into a large number of discrete chambers at limiting dilution, so that the number of target molecules and/or reference molecules in each chamber is on average about 1.59. Alternatively, the number of target molecules and/or reference molecules in a chamber may be between 0-3. In one aspect, a pre-amplification reaction may be performed on the biological sample before the biological sample is subsequently distributed into discrete subsamples. Amplification (i.e., creation of numerous essentially identical copies, for instance using polymerase chain reaction or rolling circle amplification) is carried out on a nominally single, starting molecule, where a number of individual molecules are each isolated in a separate reaction area. It is contemplated that numerous reaction areas will be used, to produce higher statistical significance. Each reaction area (well, chamber, bead, emulsion, etc.) will have either a negative result, if no starting molecule is present, or an amplification, for purposes of detection, if the targeted starting molecule is present. By analyzing the number of positive reactions, insight into the number of starting molecules is obtained. The precision, dynamic range, and sensitivity of this technique scale with the total number of chambers, becoming increasingly more powerful as the number of chambers is increased.

A number of methodologies for digital PCR exist. For example, emulsion PCR has been used to prepare small beads with clonally amplified DNA—in essence, each bead contains one type of amplicon of digital PCR. This is further described in Dressman et al. (2003). Fluorescent probe-based technologies, which can be performed on the PCR products “in situ” (i.e., in the same wells), are particularly well suited for this application. This method is described in detail in Vogelstein and Kinzler (1999), and U.S. Pat. No. 6,440,705, contains a more detailed description of this amplification procedure. The polony technique referenced below may also be used in a digital manner. These amplifications may be carried out in an emulsion or gel, on a bead or in a multiwell plate. What is advantageous is that about one molecule on average or no molecule be present in a number of reactions, such that the number of positive reactions is indicative of the number of molecules present in a sample. Accordingly, it is understood that a large number of emulsions, isolated individual molecules in a gel, beads, wells, etc. are used.

As used herein, the term “digital PCR” also includes microfluidic-based technologies where channels and pumps are used to deliver molecules to a number of chambers. A suitable microfluidic device is produced by Fluidigm Corporation™, termed the Digital Isolation and Detection IFC (integrated fluid circuit). Further description of such a device may be found in U.S. Pat. No. 6,408,878. A suitable device is also described in U.S. Pat. No. 6,960,437, which describes a microfluidic device capable of supporting multiple parallel nucleic acid amplifications and detections. One exemplary microfluidic device for conducting thermal cycling reactions includes in the layer with the flow channels a plurality of sample inputs, a mixing T-junction, a central circulation loop (i.e., the substantially circular flow channel), and an output channel. The intersection of a control channel with a flow channel can form a microvalve. This is so because the control and flow channels are separated by a thin elastomeric membrane that can be deflected into the flow channel or retracted therefrom. Deflection or retraction of the elastomeric membrane is achieved by generating a force that causes the deflection or retraction to occur. In certain systems, this is accomplished by increasing or decreasing pressure in the control channel as compared to the flow channel with which the control channel intersects. However, a wide variety of other approaches can be utilized to actuate the valves including various electrostatic, magnetic, electrolytic and electrokinetic approaches. Another microfluidic device, adapted to perform PCR reactions, and useful in the present methods, is described in US 2005/0252773 by McBride et al., published Nov. 17, 2005, and entitled “Thermal reaction device and method for using the same.”

As used herein, the term “discrete subsample” as used herein refers to a division of a biological sample containing on average about 1.59 target molecules or reference molecules.

As used herein, the term “probe” or “hybridization probe” refers to a short nucleic acid sequence that is used to detect in a nucleic acid sample the presence or absence of nucleic acid sequences that are complementary to the probe sequence. Thus, a probe will hybridize to nucleic acid sequences by probe-target base pairing. Probes are generally labeled, for instance with a radioactive or fluorescent tag, to allow detection of the hybridized nucleic acid sequence. For instance, one particularly useful type of probe is a “FRET” probe. A FRET probe, or fluorescence energy resonance transfer probe, comprises two fluorophores placed in close proximity to each other, for instance attached to each end of a sequence specific hybridization probe. Fluorophores are chosen so that the emission spectrum of one overlaps significantly with the excitation spectrum of the other. During FRET, the donor fluorophore, excited by a light source, transfers its energy to an acceptor fluorophore when positioned in the direct vicinity of the donor. Thus, the acceptor fluorophore quenches the donor fluorophore, and is sometimes referred to as the quencher. The acceptor fluorophore emits light of a longer wavelength, which is detected in specific channels. The light source cannot excite the acceptor fluorophore. In the context of nucleic acid 5 amplification, FRET probes utilize the 5′-3′ exonuclease activity of certain polymerases, which, during the amplification procedure, will cleave the FRET probe that is hybridized to the sequence to be amplified, resulting in the donor and acceptor (quencher) fluorophores no longer being directly juxtaposed—the donor fluorophore is then able to emit light, which is detected as a “positive” signal. The term “locked nucleic acid” or “LNA” refers to chemically modified RNA nucleotides having a bridge connecting 2′ and 4′ carbons. These nucleotides preserve the specificity of base pairing and increase pair stacking, resulting in more stable duplexes with significantly higher melting temperatures. A full LNA 9-mer hybridization probe has a melting temperature of approximately 60° C., allowing for the design of very short fluorescent probes. FRET and template extension reactions utilize a primer labelled with one member of a donor/acceptor pair and a nucleotide labelled with the other member of the donor/acceptor pair. Prior to incorporation of the labelled nucleotide into the primer during a template-dependent extension reaction, the donor and acceptor are spaced far enough apart that energy transfer cannot occur. However, if the labelled nucleotide is incorporated into the primer and the spacing is sufficiently close, then energy transfer occurs and can be detected. These methods are particularly useful in conducting single base pair extension reactions in the detection of single nucleotide polymorphisms and are described in U.S. Pat. No. 5,945,283 and PCT Publication WO 97/22719.

A Scorpion™ probe/primer (Biosearch Technologies™) consists of a specific probe sequence that is held in a hairpin loop configuration by complementary stem sequences on the 5′ and 3′ sides of the probe. The fluorophore attached to the 5′-end is quenched by a moiety joined to the 3′-end of the loop. The hairpin loop is linked to the 5′-end of a primer via a PCR stopper. After extension of the primer during PCR amplification, the specific probe sequence is able to bind to its complement within the same strand of DNA. This hybridisation event opens the hairpin loop so that fluorescence is no longer quenched and an increase in signal is observed. The PCR stopper prevents read-through, which could lead to opening of the hairpin loop in the absence of the specific target sequence. The Scorpion™ detection method is described, for example, by Thelwell et al., 2000 and Solinas et al., 2001. Scorpion™ primers are fluorogenic PCR primers with a probe element attached at the 5′-end via a PCR stopper. They are used in real-time amplicon-specific detection of PCR products in homogeneous solution. Two different formats are possible, the “stem-loop” format and the “duplex” format. In both cases the probing mechanism is intramolecular. The basic elements of Scorpion™ primers in all formats are: (i) a PCR primer; (ii) a PCR stopper to prevent PCR read-through of the probe element; (iii) a specific probe sequence; and (iv) a fluorescence detection system containing at least one fluorophore and quencher. After PCR extension of the Scorpion™ primer, the resultant amplicon contains a sequence that is complementary to the probe, which is rendered single-stranded during the denaturation stage of each PCR cycle. On cooling, the probe is free to bind to this complementary sequence, producing an increase in fluorescence, as the quencher is no longer in the vicinity of the fluorophore. The PCR stopper prevents undesirable read-through of the probe by Taq DNA polymerase.

A molecular bacon probe consists of a probe flanked by a hairpin loop that holds a fluorophore and quencher in close proximity until specific binding of the probe to its target opens out the structure, producing a fluorescent signal. With molecular beacons, a change in conformation of the probe as it hybridizes to a complementary region of the amplified product results in the formation of a detectable signal. The probe itself includes two sections: one section at the 5′ end and the other section at the 3′ end. These sections flank the section of the probe that anneals to the probe binding site and are complementary to one another. One end section is typically attached to a reporter dye and the other end section is usually attached to a quencher dye. In solution, the two end sections can hybridize with each other to form a hairpin loop. In this conformation, the reporter and quencher dye are in sufficiently close proximity that fluorescence from the reporter dye is effectively quenched by the quencher dye. Hybridized probe, in contrast, results in a linearized conformation in which the extent of quenching is decreased. Thus, by monitoring emission changes for the two dyes, it is possible to indirectly monitor the formation of amplification product. Probes of this type and methods of their use are described further, for example, by Piatek et al., 1998; Tyagi and Kramer, 1996; Tyagi, et al., 1998.

Another method of detection is by way of invader assays (Third Wave Technologies™ Madison, Wis.), which are used particularly for SNP genotyping and utilize an oligonucleotide, designated the signal probe, that is complementary to the target nucleic acid (DNA or RNA) or polymorphism site. A second oligonucleotide, designated the Invader Oligo, contains the same 5′ nucleotide sequence, but the 3′ nucleotide sequence contains a nucleotide polymorphism. The Invader Oligo interferes with the binding of the signal probe to the target nucleic acid such that the 5′ end of the signal probe forms a “flap” at the nucleotide containing the polymorphism. This complex is recognized by a structure specific endonuclease, called the Cleavase enzyme. Cleavase cleaves the 5′ flap of the nucleotides. The released flap binds with a third probe bearing FRET labels, thereby forming another duplex structure recognized by the Cleavase enzyme. This time, the Cleavase enzyme cleaves a fluorophore away from a quencher and produces a fluorescent signal. For SNP genotyping, the signal probe will be designed to hybridize with either the reference (wild type) allele or the variant (mutant) allele. Unlike PCR, there is a linear amplification of signal with no amplification of the nucleic acid. Further details sufficient to guide one of ordinary skill in the art are provided by, for example, Neri et al., 2000 and U.S. Pat. No. 6,706,471.

Other methods of detection are also known to those persons skilled in the art and include, without limitation: High Resolution Melting in association with dyes including LC Green™ SYT09™, Eva Green™ BEBO™ and other suitable dyes; the use of SYBR Green™ the use of BOXTO™; the use of Taqman Probes™ the use of LightCycler Hybridisation™ probes; the use of the Universal ProbeLibrary™; the use of LNA Primers™ the use of Displacing probes; the use of Light-Up Probes™; the use of a Q Zyme Assay™; Amplifluor Quantitative PCR Detection System™ LUX™ primers; and the iCycler Detection System™.

As used herein, the term “hydrolysis probe” means a probe which is used to generate a signal during an amplification reaction as a result of hydrolysis or other cleavage of the probe. It typically involves a homogeneous 5′-nuclease assay (e.g., the nuclease activity of a DNA polymerase used in PCR), since a single 3′-non-extendable (due to phosphorylation) probe, which is cleaved during PCR amplification, is used to detect the accumulation of a specific target DNA sequence. This single hydrolysis probe contains two labels in close proximity to each other: a fluorescent reporter dye at the 5′-end and a (fluorescent or dark) quencher label at or near the 3′-end. When the probe is intact, the fluorescent signal is almost completely suppressed by the quenching label.

When the probe is hybridized to its target sequence, it is cleaved by the 5′-3′ exonuclease activity of a polymerase, such as the FastStart™ Taq DNA Polymerase, which “unquenches” the fluorescent reporter dye. During each PCR cycle, more of the released fluorescent dye accumulates, boosting the fluorescent signal. In an embodiment, the probe binds to a specified strand along its length, as in a Taqman™ probe. A Taqman™ oligonucleotide probe is labelled at one end with a fluorophore and at the other end with a fluorescence quencher. When the probe binds to the target site in a PCR product the 5′-3′ exonuclease activity of Taq DNA polymerase cleaves it between the fluorophore and quencher, thereby producing an increase in fluorescence.

As used herein, the term “target probe or reference probe” refers to a molecule that can be used to report the presence of an amplified target or reference molecule, respectively. The probe may include hydrolysis probes, or molecular beacon, Scorpion™ or other probes generating a signal upon amplification. In one aspect, for example embodiments utilizing Scorpion™ probes, a probe may consist of a combination probe and primer molecule. In another aspect, a probe may consist of oligonucleotide probes distinct from a target or reference primer, such as hydrolysis probes. In all aspects, the number of target probes or reference probes will be less than the number of target or reference sites, respectively.

As used herein, the term “probe target sequence” or “probe reference sequence” refers to a nucleotide sequence of a target or reference molecule that is recognized by a target or reference probe. The probe target or probe reference sequence may be common to some or all target or reference molecules. At least two target molecules, corresponding to different target sites, will share a common probe target sequence, and at least two reference molecules, corresponding to different reference sites, will share a common probe reference sequence. The probe target sequence or probe reference sequence may be naturally present on the target or reference molecules in the biological sample (i.e., an “internal probe target sequence” or “internal probe reference sequence”). For example, a common internal probe target sequence or common internal probe reference sequence may correspond to a naturally occurring repetitive nucleic acid sequence found on the target source or reference source. Alternatively, the probe target sequence may be introduced to the target or reference molecules during a pre-amplification step with pre-amplification primers.

As used herein, the term “amplified target products” refers to nucleic acid molecules produced by digital amplification of a target molecule with a target primer pair.

As used herein, the term “amplified reference products” refers to DNA molecules produced by digital amplification of a reference molecule with a reference primer pair.

As used herein, the term “pre-amplification” refers to a DNA replication step performed on a biological sample with target and reference pre-amplification primer pairs prior to distributing the biological sample into discrete subsamples for digital amplification. Pre-amplification may be performed with a limited number of cycles so as to avoid biased replication of individual target molecules. In one aspect, a pre-amplification step may serve to synthesize target and reference molecules that are more amenable to amplification by target and reference primers. In another aspect, a pre-amplification step may be performed using pre-amplification primers designed to introduce the sequences on a target or reference molecule to which a target primer or reference primer may anneal. Such sequences may comprise universal non-specific primer sequences. In yet another aspect, a pre-amplification step may be performed with pre-amplification primers designed to introduce a probe target sequence or a probe reference sequence to a target molecule or reference molecule.

As used herein, the term “nucleic acid” as used herein includes any nucleic acid, and may be a deoxyribonucleotide or ribonucleotide polymer in either single or double-stranded form. A “polynucleotide” or “nucleotide polymer” as used herein may include synthetic or mixed polymers of nucleic acids, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

As used herein, the term “purine” refers to a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′ 5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain. A “pyrimidine” is a single-ringed, organic base that forms nucleotide bases, such as cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

As used herein, the term “rolling circle amplification”, also referred to as “rolling circle chain reaction”, “RCA”, or “RCCR” refers to a method whereby amplification occurs in a continuous fashion using short circular DNA as a template, resulting in the production of a concatameric nucleic acid containing multiple copies of the target sequence arranged in a head-to-tail format. This reaction may occur at isothermal conditions. The technique is based upon the rolling circle replication process that various biological organisms use to replicate their genomes. A rolling circle amplification reaction may be accomplished in several different ways, for instance as described in Demidov, (2005) Expert Rev. Mol. Diagn. In certain methods, a circular nucleic acid template must first be created. For instance, a nucleic acid primer sequence may be designed that contains two sequence complementary regions that flank the target sequence. These two sequence complementary regions are analogous to a pair of primers used in PCR. However, in these “padlock probes”, also referred to as “molecular inversion probes”, the complementary regions are arranged on the probe at each end of the probe in an opposite orientation, rather than facing each other, as they would in PCR. The primer would also contain an intervening sequence to separate the complementary regions. Hybridization of the molecular inversion probe to the template results in each complementary region binding to each flanking site in its proper orientation—ie., facing each other. For this to happen, one of the primers must “flip”, which causes the molecular inversion probe to form a loop. Subsequent extension of the target sequence between the flanking sites using a polymerase, followed by ligation using a ligase would result in a circular nucleic acid containing the target sequence, which could be used for subsequent rolling circle amplification. More specifically, padlock probes (PLPs) are long (e.g., about 100 bases) linear oligonucleotides. The sequences at the 3′ and 5′ ends of the probe are complementary to adjacent sequences in the target nucleic acid. In the central, noncomplementary region of the PLP there is a “tag” sequence that can be used to identify the specific PLP. The tag sequence is flanked by universal priming sites, which allow PCR amplification of the tag. Upon hybridization to the target, the two ends of the PLP oligonucleotide are brought into close proximity and can be joined by enzymatic ligation. The resulting product is a circular probe molecule catenated to the target DNA strand. Any unligated probes (i.e., probes that did not hybridize to a target) are removed by the action of an exonuclease. Hybridization and ligation of a PLP requires that both end segments recognize the target sequence. In this manner, PLPs provide extremely specific target recognition.

Other methods for producing appropriate templates for use in rolling circle amplification are described in the art. Amplification from such circular templates may then be achieved using a single sequence specific primer, along with other components necessary for nucleic acid extension, such as polymerase, free nucleotides, and appropriate buffers. Alternatively, the circular templates may be amplified using traditional PCR, with two sequence specific primers.

As used herein, the term “template” refers to a nucleic acid which is to be amplified, for instance via PCR or rolling circle amplification. The template, for instance a DNA template, is added to the amplification reaction as the starting material from which the amplification reaction proceed, leading the amplification of a defined region of the template. The amplification primers used in the reaction are generally designed from the template nucleic acid sequence such that they bind to specific regions of the template, as described elsewhere in the application.

As used herein, the term “primer”, or “PCR primer” or “amplification primer” refers to short nucleic acid fragments which hybridize to complementary nucleic acid sequences and which may serve as starting points for DNA replication—i.e., synthesis of new nucleic acid fragments, for instance by extension with DNA polymerase. DNA polymerase is only able to add new nucleotides in a 5′ to 3′ direction, meaning that new nucleotides are added to the 3′ end of existing nucleic acid fragments. Thus, a sequence-specific PCR primer may be designed to hybridize to a specific complementary sequence such that the nucleic acid sequence that is directly 3′ to primer sequence may be replicated in an appropriate reaction, for instance in a polymerase chain reaction. In PCR, a pair of opposing PCR primers may be utilized to define the boundaries of the nucleic acid fragment that is to be amplified—in such case, the 5′ end of each PCR primer in the primer pair will generally define the beginning and end of the amplified nucleic acid fragment. In the context of certain embodiments, it is important to note that during the PCR amplification process, the PCR primers are themselves incorporated into the amplified nucleic acid fragment. PCR primers may be optimized to maximize their efficiency in an amplification reaction, and such optimization procedures are described in the art. For instance, one may vary the primer length and composition, to optimize the annealing temperature of the primers, and to minimize the effects of secondary structure formation and the like. The composition of the primer may be varied by choosing difference sequence specific regions of the template from which to amplify—i.e., if one desires to amplify a specific region of the template, the flanking regions of that region may be analyzed to find the most ideal hybridization sequence from which one would derive the complementary primer sequence. The inventors herein provide novel compositions and methods relating to PCR and PCR primers with application to the hybridization to, amplification of, and/or quantification of fetal and/or maternal chromosomal DNA, and for the detection or identification of fetal chromosomal abnormalities.

“Multiplex analysis” as used herein refers to the simultaneous analysis of target and/or reference sites in a single reaction. A multiplex analysis or reaction may be referred to as “multiplexing”. A multiplex analysis of nucleic acids using an amplification procedure would be called a “multiplex amplification”. For instance, in multiplex PCR, a number of different primer sets may be included in the same reaction, leading to simultaneous amplification of multiple genetic loci. Subsequent analysis or detection of the different amplified genetic loci generally requires additional techniques, for instance separation by gel electrophoresis. One of the advantages of digital amplification, however is rapid scoring of the amplification product, and this advantage would be lost if a downstream separation step were added. Therefore, issues of non-specific noise are highly relevant.

Multiplex amplification also has further inherent limitations, and generally requires significant optimization in order to achieve adequate results. Moreover, these limitations generally increase with the number of additional loci that are amplified or analyzed. For instance, each primer pair in the reaction should have relatively similar properties such as hybridization stringency, amplification efficiency, etc., in order to achieve optimal and consistent results. In addition to this, the inclusion of a plurality of probes into a single reaction can significantly increase the amount of non-specific primer hybridization and amplification, as well as the amount of primer-primer cross-reaction and cross-hybridization. When multiplexing in normal PCR, much of this non-specific “noise” can be removed by subsequent separation of the PCR products, for instance by gel electrophoresis or using microarrays.

When using multiple sets of primer sets and probes in the same reaction, where each probe is the same color for each amplified locus, one runs into the problem of very high background signal. This is due to the requirement to keep the concentration of each locus-specific probe at a high enough level to ensure binding to the specific amplified nucleic acid fragment. Since each probe has its own endogenous background signal, the cumulative effect of each of the probes would quickly quench the true signal from the reaction. In addition, the high number of probe sequences increases the nucleic acid load in each reaction, leading to increased nucleic acid cross-reactivity, non-specific priming, and the like. An alternate method for multiplexing using different probe colors for each genetic locus is limited by the number of different probes colors available, and one would quickly run out of probes. Such an alternate method would also suffer the same limitations of high nucleic acid load in each reaction, due to the inclusion of multiple probes in each reaction.

The compositions and methods described herein allow for increased sensitivity of existing digital amplification methods by introducing multiplexing into the standard digital amplification methodology. Typically, when utilizing digital amplification for quantification of chromosomes in a biological sample, for instance for detecting fetal chromosomal aneuploidy in a maternal blood sample, a single primer pair would be utilized for each chromosome that is being quantified. In such a fashion, the presence of that chromosome in a digital amplification microchamber would cause amplification via the chromosome specific primer pair, resulting in a positive signal from that microchamber. The methods described herein, take advantage of the fact that chromosomes found within biological samples, for instance maternal and fetal chromosomes found in maternal blood, are generally significantly fragmented—DNA circulating in the plasma, for instance, is degraded into short fragments having characteristic lengths of 100 to 1000 nucleotides so that each copy of the genomic DNA is represented by a large number of unique sequences that are characteristic of their chromosome of origin. Multiple primer pairs are utilized herein for a given chromosome to be quantified, such primer pairs being designed to hybridize to and amplify different regions of the same chromosome in a multiplex fashion. Each chromosome fragment may be independently present in a digital amplification microchamber, thus each chromosome fragment provides a chromosomal representative which may be counted for quantification of the chromosome. Thus, the multiplexed targeting of many sequences that are sufficiently spaced from one another, and hence are statistically independent during sampling, provides a means of increasing the effective number of chromosomal equivalents in a sample, resulting in a commensurate reduction in required sample volume and concentration. By utilizing multiple primer pairs which are multiplexed in the same digital amplification, and which each bind at various points across the chromosome, then many more chromosomal fragments may be counted from a single digital amplification, thus increasing the sensitivity and accuracy of the chromosomal quantification, as compared to chromosomal quantification using digital amplification with a single primer set for a given chromosome. In various embodiments the methods allow for accurate and sensitive chromosomal analysis from much smaller biological samples, which may have significant practical advantages. For instance, the compositions and methods described herein may allow identification or detection of fetal chromosomal aneuploidy from maternal blood samples of limited size, for instance 5 mL of maternal blood, or smaller.

The highly sensitive quantification of chromosomes in maternal samples, for instance blood samples, may allow for accurate comparison of the amount of a target chromosome, for instance chromosome 21, to a non-target reference chromosome, for instance chromosome 9. By using optimized chromosome-specific multiplex primer sets, such as those described herein, one may establish a “normal” ratio of quantified target:non-target chromosomes (i.e., chr21:chr9)—in other words, the ratio that would be expected in maternal samples from subjects with no fetal chromosomal aneuploidy. Deviations from “normal” in the ratio of target to non-target chromosomes in a given maternal blood sample from a subject would thus be indicative of the presence of a fetal aneuploidy in the subject—this is due to over- or under-representation of the target chromosome in the maternal sample due to the presence or absence of a chromosome in the fetus of that subject.

The disclosed methods may further be used for the non-invasive determination of fetal genotype in multi-gestational pregnancy including at least one male fetus. In a mono-gestational pregnancy involving a male fetus, the theoretical ratio of X:Y chromosomes in maternal blood with a fetal DNA concentration of 10% would be 19:1. In a multi-gestational pregnancy involving one male fetus and one female fetus, the theoretical ratio of X:Y chromosomes in maternal blood with a fetal DNA concentration of 10% would be 39:1 (assuming equal contribution of DNA by each fetus). In a multi-gestational pregnancy involving two male fetuses and one female fetus, the theoretical ratio of X:Y chromosomes in maternal blood with a fetal DNA concentration of 10% would be 29:1. In a multi-gestational pregnancy involving two female fetuses and one male fetus, the theoretical ratio of X:Y chromosomes in maternal blood with a fetal DNA concentration of 10% would be 59:1. These ratios increase with decreasing concentration of fetal DNA in the maternal blood. Accordingly, the methods disclosed herein can be used to make fetal sex determinations early in pregnancy when the fetal DNA concentration in maternal blood is low.

The disclosed methods may further be used for the non-invasive detection of fetal Mendelian disorders where both parents are carriers of the same recessive deleterious allele. Provided with the existence of an adequate number polymorphisms, such as SNPs, between the chromosomal regions on which the maternal dominant allele and the recessive allele are located, and which are tightly linked on to the gene of interest, the disclosed methods could be used to determine the ratio of the dominant allele to the recessive allele in the maternal blood. Assuming a fetal DNA concentration of 10% in the maternal blood, the ratio of recessive allele to dominant allele in the case of a homozygous recessive fetus would be 1.22. This ratio would increase with decreasing concentration of fetal DNA in the maternal blood. Accordingly, the methods disclosed herein can be used to make a diagnosis early in pregnancy when the fetal DNA concentration in maternal blood is low.

The disclose methods may further be used for the non-invasive detection or surveillance of a chromosomal abnormality that may be indicative a pathology, such as cancer or a pre-cancerous condition, in a subject. For example, the disclosed methods may be used to detect the presence of cells in the body of a subject which comprise a gain or loss of a defined target source, or portion thereof. Various biological samples may be taken from the subject, depending upon the pathology, including but not limited to blood samples, urine samples, stool samples, vaginal secretions or other body exudates. Overrepresentation, or underrepresentation, of the target source relative to the reference source(s) would be indicative of the presence of cells in the body which carry a chromosomal abnormality associated with the target source. Moreover, the degree of overrepresentation, or underrepresentation, may indicate the abundance of cells which comprise a chromosomal abnormality. Accordingly, the disclosed methods may further be used to monitor the progression of a pathological condition, or evaluate the efficacy treatments for the condition.

### Sample Preparation

In various embodiments, one may also use samples including saliva, urine, tear, vaginal secretion, breast fluid, breast milk, feces, umbilical cord blood, chorionic amniotic fluid, embryonic tissue, lymph fluid, cerebrospinal fluid, mucosa secretion, peritoneal fluid, ascitic fluid, or body exudates (including sweat). In the case of fetal chromosomal abnormality detection, however, the preferred starting material is maternal peripheral venous blood.

### Isolation of Blood

In order to obtain sufficient DNA for testing, it is preferred that 10-20 ml of blood be drawn, in order to obtain about at least 10,000 genome equivalents of total DNA. This sample size is based on an estimate of fetal DNA being present as roughly 25 genome equivalents/ml of maternal plasma in early pregnancy, and a fetal DNA concentration of about 3.4% of total plasma DNA. However, less blood may be drawn for a genetic screen where less statistical significance is required, or the DNA sample is enriched for fetal DNA.

Blood may be collected by any method or process that results in a substantial increase in the ratio of fetal DNA/maternal DNA in the resulting serum or plasma after appropriate processing. As used herein, a substantial increase in the ratio of fetal DNA/maternal DNA is that which can be detected by the methods as described herein. Such methods or processes typically result in a substantial increase in the ratio of fetal DNA/maternal DNA of about 5%, 10%, 15%, 20%, 30%, 50%, 70%, 80%, 100% or more of the ratio of fetal DNA/maternal DNA found in blood samples collected by standard procedures.

In other embodiments, blood is collected by any method or process that results in a substantial increase in the amount of free fetal DNA compared to the amount of total DNA recovered or detected in the resulting serum or plasma after processing. Such methods or processes typically result in a substantial increase so the fetal DNA recovered or detected is about 10%, 15%, 20%, 25%, 30%, 40%, 50% or more of the total DNA recovered or detected in the processed plasma or serum sample.

The methods or processes of collecting blood samples may also include other steps that result in lessened or reduced cell lysis. For instance, blood collection devices may be modified to decrease cell lysis due to sheer forces in the collection needle, syringe or tubes used. For instance, needles of large gauge may be employed to reduce cell sheering or vacutainer tubes may be modified to reduce the velocity of blood flow.

United States Patent Application 20040137470 describes an enrichment procedure for fetal DNA in which blood from pregnant women is collected into 9 ml EDTA Vacuette™ tubes (catalog number NC9897284) and 0.225 ml of 10% neutral buffered solution containing formaldehyde (4% w/v), is added to each tube, and each tube gently is inverted. The tubes are stored at 4.degree° C. until ready for processing.

Agents that impede cell lysis or stabilize cell membranes added to the maternal blood to reduce maternal cell lysis including but not limited to aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188™.

Flow cytometry techniques can also be used to enrich fetal cells (Herzenberg et al., PNAS, 76: 1453-1455 (1979); Bianchi et al., PNAS, 87: 3279-3283 (1990); Bruch et al., Prenatal Diagnosis 11: 787-798 (1991)). U.S. Pat. No. 5,432,054 also describes a technique for separation of fetal nucleated red blood cells, using a tube having a wide top and a narrow, capillary bottom made of polyethylene. Centrifugation using a variable speed program results in a stacking of red blood cells in the capillary based on the density of the molecules. The density fraction containing low-density red blood cells, including fetal red blood cells, is recovered and then differentially hemolyzed to preferentially destroy maternal red blood cells. A density gradient in a hypertonic medium is used to separate red blood cells, now enriched in the fetal red blood cells from lymphocytes and ruptured maternal cells. The use of a hypertonic solution shrinks the red blood cells, which increases their density, and facilitates purification from the more dense lymphocytes. After the fetal cells have been isolated, fetal DNA can be purified using standard techniques in the art.

### Isolation of Plasma

Any method may be used to isolate plasma from the cell components of blood after collection but methods are preferred wherein cell lysis is substantially prevented, reduced or inhibited. The blood may be stored at 4° C. until processing. Methods for isolation of the plasma may be implemented to reduce the amount of maternal cell lysis.

Collection tubes may be spun at 1000 rpm for ten minutes in a centrifuge with braking power and acceleration power set at zero to substantially prevent, reduce or inhibit cell lysis and or mixing of blood cell components into the plasma. The tubes may be spun a second time at 1000 rpm for ten minutes with braking power and acceleration power set to zero. The supernatant (i.e. plasma) of each sample may be transferred to a new tube and spun at 3000 rpm for ten minutes with the brake and acceleration power set at zero. The supernatant (i.e. plasma) of each sample may be collected using procedures to substantially prevent mixing of cell components into the plasma. A percentage of the supernatant can be left in the tube including but not limited to 0.001-1%, 1-10%, 10-20%, 20-30%, 30-40%, 40-50%, 50-60%, 60-70%, 70-80% or greater than 80%. In this example, about 0.5 ml of the supernatant was left in the tube to ensure that the buffy-coat was not disturbed. The supernatant may be transferred to a new tube and stored at −80° C.

Enrichment of DNA from Plasma

The maternal blood may be processed to enrich the fetal DNA concentration in the total DNA, as described in Li et al., supra. Briefly, circulatory DNA is extracted from 5- to 10-mL maternal plasma using commercial column technology (Roche High Pure Template DNA Purification Kit™; Roche™, Basel, Switzerland) in combination with a vacuum pump. After extraction, the DNA is separated by agarose gel (1%) electrophoresis (Invitrogen™), and the gel fraction containing circulatory DNA with a size of approximately 300 by is carefully excised. The DNA is extracted from this gel slice by using an extraction kit (QIAEX II Gel Extraction Kit™; Qiagen™) and eluted into a final volume of 40-.mu·L sterile 10-mM trishydrochloric acid, pH 8.0 (Rocher™).

DNA may be concentrated by known methods, including centrifugation and various enzyme inhibitors. The DNA is bound to a selective membrane (e.g., silica) to separate it from contaminants. The DNA is preferably enriched for fragments circulating in the plasma, which are less than 1000 base pairs in length, generally less than 300 bp. This size selection is done on a DNA size separation medium, such as an electrophoretic gel or chromatography material. Such a material is described in Huber et al., “High-resolution liquid chromatography of DNA fragments on non-porous poly(styrene-divinylbenzene) particles,” Nucleic Acids Res. 1993 Mar. 11; 21(5): 1061-1066, gel filtration chromatography, TSK gel, as described in Kato et al., “A New Packing for Separation of DNA Restriction Fragments by High Performance Liquid Chromatography,” J. Biochem, 1984, Vol. 95, No. 1 83-86.

In addition, enrichment may be accomplished by suppression of certain alleles through the use of peptide nucleic acids (PNAs), which bind to their complementary target sequences, but do not amplify.

### Isolation of DNA

Any method of DNA isolation can be used including cesium chloride gradients, gradients, sucrose gradients, glucose gradients, centrifugation protocols, boiling, Qiagen™ purification systems, QIA™ DNA blood purification kit, HiSpeed Plasmid Maxi Kit™, QIAfilter™ plasmid kit, Promega™ DNA purification systems, MangeSil Paramagnetic Particle™ based systems, Wizard SV™ technology, Wizard Genomic™ DNA purification kit, Amersham™ purification systems, GFX™ Genomic Blood DNA purification kit, Invitrogen Life Technologies™ Purification Systems, CONCERT™ purification system, Mo Bio Laboratories™ purification systems, UltraClean BloodSpin Kits™, and UlraClean Blood DNA Kit™. A person of ordinary skill in the art understands that the manufacturer's protocols can modified to increase the yield of DNA.

### EXAMPLES

The following Examples are provided for illustrative purposes, and are not intended to be limiting.

### Example 1

### Calculation of Digital PCR Precision

The theoretical precision of digital PCR analysis depends on the total number of chambers (N) and the expected number of molecules per chamber (A). In the case of NA >>1 the number of molecules present in each chamber is a random variable k that is well described as an independent Poisson process. The probability of detecting at least one molecule is P(k>0)=1−e^{-λ}. Let x be a random variable describing the number of chambers having at least one molecule in an array of N chambers. P(x) is therefore given by a binomial distribution with mean N(1−e^{-λ}) and variance σ²=N(e^{-λ}−e^{-2λ}). Under the condition of large N this is well approximated as a Gaussian distribution:

$$P ( x ) = \frac{1}{\sqrt{2 π N ( {e}^{- λ} - {e}^{- 2 λ} )}} exp ( - \frac{{( x - N ( 1 - {e}^{- λ} ) )}^{2}}{2 N ( {e}^{- λ} - {e}^{- 2 λ} )}$$

We define the precision of a digital PCR measurement as the minimum difference in concentration Δλ that can be reliably detected with less than 1% false positive and less than 1% false negative. This corresponds to a 4.6 σ separation in the mean for two Guassian distributions. For the case of small differences in concentration we linearize the ratio of the expectation of x and the standard deviation to obtain the following condition:

$$\begin{matrix}\frac{\partial }{\partial λ} ( N ( 1 - {e}^{- λ} ) ) Δ λ = N {e}^{- λ} Δ λ \\ = 4.6 ( {{N}^{1 / 2} ( {e}^{- λ} - {e}^{- 2 λ} )}^{1 / 2} )\end{matrix}$$

From which we obtain the measurement precision as:

Δλ/λ=4.62λ⁻¹N^{-1/2}(e^{λ}−1)^{1/2}

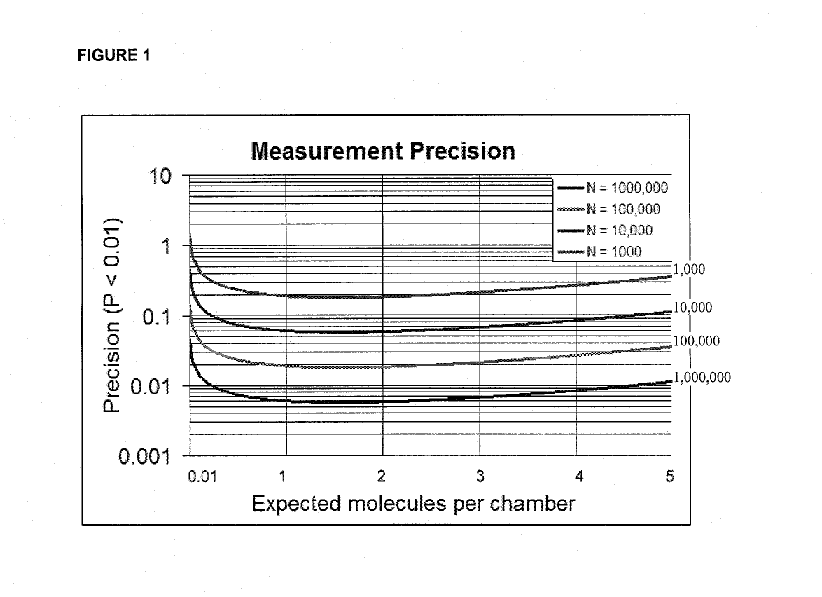

The FIG. 1 shows this function plotted for N=1000, 10000, 100000, and 1000000 across a range of λ from 0.01 to 5.

Minimizing this function with respect to λ gives the optimal concentration to achieve maximum precision. This is approximately 1.59 molecules per discrete subsample (for example, a chamber).

### Example 2

### Calculation of the Separation in the Measured Mean of Two Alleles Varying by 1% Using Digital PCR as a Function of the Number of Discrete Subsamples (for Example, Chambers)

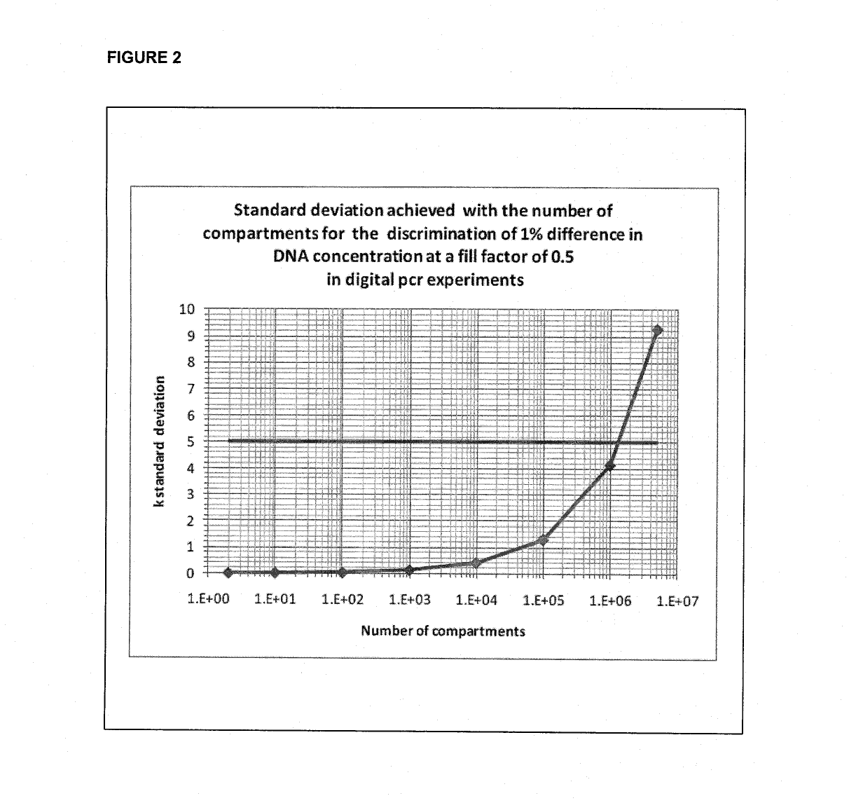

FIG. 2 shows a numerical calculation of the separation in the measured mean of two alleles varying by 1% using digital PCR as a function of the number of chambers. Difference is normalized by the expected standard deviation (sigma) as determined by the combined effect of 5 stochastic Poisson variation (curved line). The calculation was performed for template concentration corresponding to positive amplification in 50% of wells. 5 sigma separation (horizontal line) is achieved at approximately 1,000,000 chambers. Standard deviation achieved with the number of compartments for the discrimination of 1% difference in DNA concentration at a fill factor of 0.5 in digital PCR experiments.

### Example 3

### Theoretical Calculations of Sample Molecule Content and Blood Sample Volume

Fetal DNA is reported to occur in maternal blood and represents approximately 2 to 6 percent of the total DNA present in the cell-free serum during the first trimester (Lo et al. 1997; Lo et al. 1998; Wachtel et al. 2001; Lee et al. 2002). However, later publications have suggested that the fetal contribution may be 9.7%, 9.0%, and 20.4% for the first, second, and third trimesters, respectively (Lun et al. 2008). For example, each genome copy of the T21 fraction contributes an extra copy of chromosome 21, which allows for a direct non-invasive maternal blood test by measuring the ratio of chromosome copy numbers present in maternal serum. If we assume a 2% fraction of fetal DNA in a maternal blood sample, the expected enrichment of chromosome 21 with respect to the other chromosomes in the pool is (0.02×1)/(1×2)=1%. Such a small difference in relative concentrations is undetectable by qRT-PCR which can at best reliably distinguish concentrations of ˜20%.

Lo et al. (2007) showed that 25% discrimination was achievable with 7,680 PCR reactors, which could be obtained by using 20 separate 384 well plates. A separate study using commercially available microfluidic chips, the minimum fraction of T21 DNA that could be detected with 5-sigma confidence was 20% (Fan and Quake 2007). High confidence detection of a 1% enrichment of chromosome 21 requires sampling of approximately 500,000 molecules within 1,000,000 chambers (FIG. 2). This large amount of DNA raises a practical issue in the sample volume required for testing. It is estimated that during the first trimester of pregnancy the amount of fetal DNA circulating in the maternal serum is 5000 copies/ml (Lo et al. 1998; Li et al. 2004). Accordingly, a digital assay based on a single chromosome locus would require a total volume of approximately 100 mls of blood serum (i.e. about 182 mls of blood)—an amount that is not practical for clinical screening. Keeping in mind that a unit of blood, the amount collected during a typical blood donation is about half a litre (500 mls), or one pint (473 mls). In addition, the purification and subsequent concentration of dilute genomic DNA to the required volume of ˜10 μL would introduce significant losses, further contributing to the problem.

TABLE 1 shows theoretical calculations of the estimated number of molecules in sample to achieve a high confidence detection by number of loci sampled (1-10) and based on the percentage of fetal DNA in the maternal blood sample (1%-20%) and the expected enrichment of the target chromosome (0.5%-10%).

TABLE 1
Estimated Number of Molecules in Sample

| Fraction | | | | | | |
|---|---|---|---|---|---|---|
| of DNA | Expected | | | | | |
| that is of | Enrichment | Estimated Number of Molecules in Sample | | | | |
| Fetal | of Target | to Achieve a High Confidence Detection | | | | |
| Origin | Chromosome | by Number of Loci Sampled | | | | |
| (%) | (%) | 1 | 2 | 3 | 4 | 5 |
| 1 | 0.5 | 1,590,000 | 795,000 | 530,000 | 397,500 | 318,000 |
| 2 | 1.0 | 240,000 | 120,000 | 80,000 | 60,000 | 48,000 |
| 3 | 1.5 | 100,000 | 50,000 | 33,333.33 | 25,000 | 20,000 |
| 4 | 2.0 | 56,000 | 28,000 | 18,666.67 | 14,000 | 11,200 |
| 5 | 2.5 | 36,000 | 18,000 | 12,000 | 9,000 | 7,200 |
| 6 | 3.0 | 25,000 | 12,500 | 8,333.333 | 6,250 | 5,000 |
| 7 | 3.5 | 18,000 | 9,000 | 6,000 | 4,500 | 3,600 |
| 8 | 4.0 | 13,000 | 6,500 | 4,333.333 | 3,250 | 2,600 |
| 9 | 4.5 | 11,000 | 5,500 | 3,666.667 | 2,750 | 2,200 |
| 10 | 5.0 | 8,500 | 4,250 | 2,833.333 | 2,125 | 1,700 |
| 15 | 7.5 | 3,800 | 1,900 | 1,266.667 | 950 | 760 |
| 20 | 10.0 | 2,000 | 1,000 | 666.6667 | 500 | 400 |

| Fraction | | | | | |
|---|---|---|---|---|---|
| of DNA | | | | | |
| that is of | Estimated Number of Molecules in Sample | | | | |
| Fetal | to Achieve a High Confidence Detection | | | | |
| Origin | by Number of Loci Sampled | | | | |
| (%) | 6 | 7 | 8 | 9 | 10 |
| 1 | 265,000 | 227,142.9 | 198,750 | 176,666.7 | 159,000 |
| 2 | 40,000 | 34,285.71 | 30,000 | 266,66.67 | 240,00 |
| 3 | 16,666.67 | 14,285.71 | 12,500 | 1,1111.11 | 10,000 |
| 4 | 9,333.333 | 8,000 | 7,000 | 6,222.222 | 5,600 |
| 5 | 6,000 | 5,142.857 | 4,500 | 4,000 | 3,600 |
| 6 | 4,166.667 | 3,571.429 | 3,125 | 2,777.778 | 2,500 |
| 7 | 3,000 | 2,571.429 | 2,250 | 2,000 | 1,800 |
| 8 | 2,166.667 | 1,857.143 | 1,625 | 1,444.444 | 1,300 |
| 9 | 1,833.333 | 1,571.429 | 1,375 | 1,222.222 | 1,100 |
| 10 | 1,416.667 | 1,214.286 | 1,062.5 | 944.4444 | 850 |
| 15 | 633.3333 | 542.8571 | 475 | 422.2222 | 380 |
| 20 | 333.3333 | 285.7143 | 250 | 222.2222 | 200 |

If we assume that there are 5000 copies/ml (as suggested in Lo et al. 1998; Li et al. 2004), then a digital amplification would require about 100 mls of blood serum (i.e. about 182 mls of blood) to sample 500,000 molecules. Similarly, TABLE 2 sets out the estimated number of molecules that are to be sampled against the blood serum volume and blood volume required from the maternal subject.

TABLE 2
Blood Volumes

| Number of Molecules | Blood Serum | Blood |
|---|---|---|
| to be Sampled | Volume (ml) | Volume (ml) |
| 1,000,000 | 200 | 364 |
| 500,000 | 100 | 182 |
| 333,333 | 66.7 | 121.4 |
| 250,000 | 50 | 91 |
| 200,000 | 40 | 72.8 |
| 166,667 | 33.3 | 60 |
| 142,857 | 28.6 | 52 |
| 125,000 | 25 | 45.5 |
| 111,111 | 22 | 40 |
| 100,000 | 20 | 36.4 |
| 75,000 | 15 | 27.3 |
| 50,000 | 10 | 18.2 |
| 40,000 | 8 | 14.6 |
| 30,000 | 6 | 10.9 |
| 20,000 | 4 | 7.3 |
| 10,000 | 2 | 3.64 |
| 5,000 | 1.0 | 1.82 |

### Example 4

### Multiplex PCR with Multiple chr.21 Targets Using a Single Internal Amplified Probe Sequence Results in a Linear Increase in Amplification Signal

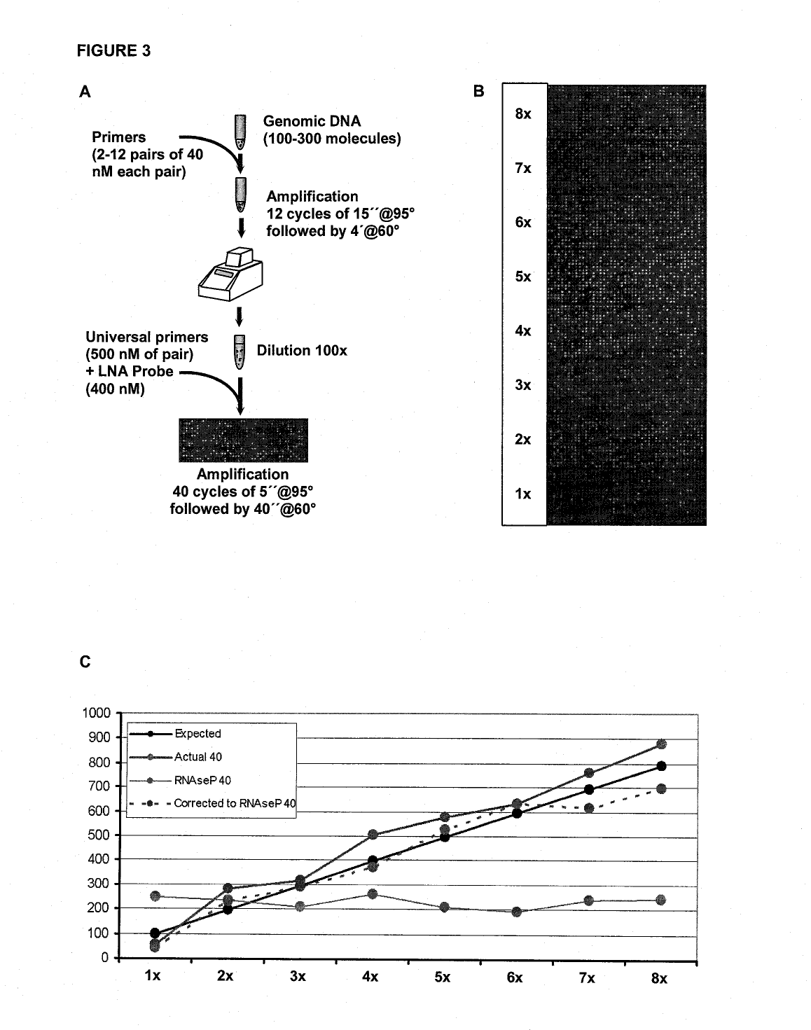

An 8 base sequence (5′-CTGGCTCC-3′) (SEQ ID NO:19) that is represented multiple times on chromosome 21 has been identified. Of these an initial panel of 8 were selected that were separated by greater than 10 Kb and 8 primer sets were designed to amplify these regions (see: TABLE 3). These primer sets have been successfully used for the multiplexed amplification of 8 target sequences with fluorescent detection using a single LNA probe labelled with 6-5 carboxyfluorescein (FAM) on the 5′ end, and containing the quencher BHQ1 on the 3′ end (FAM-CTGGCTCC-BHQ1) (SEQ ID NO:20)—results shown in FIG. 3. FIG. 3A shows the two-step amplification strategy—the first step is a pre-amplification step in which the plurality of primers (i.e., all 8 primer sets from TABLE 3, SEQ ID NOs:1-16) were mixed with the template DNA, along with the necessary amplification components (i.e., polymerase, dNTPs, Mg2+, etc.), and amplified for a limited number of cycles (for instance 12 cycles). The second step is the digital amplification step in which the product of the step 1 is mixed with the labelled hybridization probe and universal primers (for instance, SEQ ID NOs:17-18), diluted (for instance, a 100× dilution), and separated into multiple microchambers, for instance by loading into a device such as a multichamber microfluidic device. FIG. 3B shows the results of a series of digital reactions using an increasing number of primer pairs from TABLE 3. These results clearly demonstrate that the number of chambers with a positive signal increases in a linear fashion that is proportional to the number of primer pairs included in the amplification reaction. This strategy obviates the need for including multiple fluorescent probes in the assay which would result in very high fluorescent background due to incomplete quenching of uncleaved FRET probes, and the results clearly show the successful multiplex digital PCR from chromosome 21, which will allow for a significant increase in the signal that may be acquired from a limited maternal sample, for instance from a small amount of maternal blood. The primers may be optimized by testing various combinations of primer sequences, their concentration and adjusting conditions of PCR reaction (buffer and Mg2+ ion concentrations, enzyme and dNTP concentrations) to ensure precision and high signal to noise. To establish an internal control for chromosome copy number, one would design and optimize a similar multiplexed assay for a reference chromosome (for instance chromosome 9) in a similar fashion as just shown for chromosome 21, using a single LNA probe labelled with a spectrally distinct fluorophore. The use of these two optimized chromosome 21 and reference chromosome specific primer sets would allow for a quantification of both chromosome 21 and a reference chromosome from a single maternal sample, such as a blood sample, and would allow the establishment of a “normal” [chromosome 21:reference chromosome] ratio in a maternal blood sample in which there is no fetal chromosomal aneuploidy. Significant deviation from this ratio in a given maternal sample from a subject would thus be indicative of a fetal chromosomal abnormality. Over-representation of a target chromosome, in this case chromosome 21, in a sample may be due to the presence of an extra chromosome in the fetus. It may be possible to establish a range of deviation from normal, or an expected deviation from normal, that could be expected in the case of a fetal aneuploidy, by testing a population of subjects that have such fetal aneuploidy. This deviation from the normal ratio could be indicative of a fetal aneuploidy.

TABLE 3
Sequence specific, optimized primer pairs, designed to
amplify a common internal probe target sequence on
chromosome 21.
Each primer comprises a sequence-specific sequence,
non-underlined, as well as a universal primer
target sequence, underlined.

| | Primer | SEQ ID | Pair |
|---|---|---|---|
| Primer Sequence | Name | NO: | # |
| gttgtaaaacgacggccagtacggaagcagaggcttctaa | Pair1-F | 1 | 1 |
| cacaggaaacagctatgacctccgagtgttttcagatgga | Pair1-R | 2 | 1 |
| gttgtaaaacgacggccagtagcaggtggggtggattt | Pair6-F | 3 | 2 |
| cacaggaaacagctatgaccaacctgggaggcagcttag | Pair6-R | 4 | 2 |
| gttgtaaaacgacggccagtcatgtcagccgagtctcctc | Pair12-F | 5 | 3 |
| cacaggaaacagctatgacccctggcaattgtccacct | Pair12-R | 6 | 3 |
| gttgtaaaacgacggccagtgtcttcacgagcgtgcatc | Pair14-F | 7 | 4 |
| cacaggaaacagctatgaccaaacccgtgtctgactcactg | Pair14-R | 8 | 4 |
| gttgtaaaacgacggccagtgccttacatgtgaaagtgcgta | Pair31-F | 9 | 5 |
| cacaggaaacagctatgaccgctctctgaatcctgtgtggt | Pair31-R | 10 | 5 |
| gttgtaaaacgacggccagtgggcactgactggtctaagg | Pair32-F | 11 | 6 |
| cacaggaaacagctatgacccacaactccagactggctacag | Pair32-R | 12 | 6 |
| gttgtaaaacgacggccagtgagaccacgctagtcacagga | Pair33-F | 13 | 7 |
| cacaggaaacagctatgaccggtgagaagaggcagtgagc | Pair33-R | 14 | 7 |
| gttgtaaaacgacggccagtcccagggacacagctagtaagt | Pair34-F | 15 | 8 |
| cacaggaaacagctatgaccagattctgccgccatcct | Pair34-R | 16 | 8 |

Further, and with reference to FIG. 3 herein, this Figure demonstrates a digital PCR analysis of two-step amplification protocol using primers with gene specific regions and flanking universal primer sequences. Part (A) of FIG. 3 demonstrates a schematic diagram for a potential multiplex digital PCR, which includes a pre-amplification step. The pre-amplification occurs with a plurality of PCR primer pairs, and each primer pair can amplify a PCR product comprising a common internal probe sequence. Dilution of the pre-amplification products and subsequent digital PCR is then performed using a single hybridization probe (in this example, a “locked nucleic acid” (LNA) FRET probe). Part (B) of FIG. 3 shows digital micrographs of 40th PCR cycle from multiplex digital PCR with progressively higher numbers of molecules detected corresponding to increasing the number of gene-specific primer pairs (see TABLE 3) used in the pre-amplification protocol at constant template concentration. Experimental data (Part C) of a number of target sequences detected is plotted along with RNAse P control gene as a function of target regions. Pre-amplification primers at 40 nM concentration of each pair were used. Increasing signal was achieved using an increasing number of PCR primer pairs in a multiplex digital PCR. The optimal linear increase in signal that would be expected to occur upon the inclusion of each additional primer pair, in the absence of background noise, signal saturation or quenching, and the like, as shown.

Although the foregoing has been described in some detail by way of illustration and example for purposes of clarity of understanding, it will be readily apparent to those of skill in the art in light of the teachings herein that changes and modification may be made thereto without departing from the spirit or scope of the appended claims.

### BIBLIOGRAPHY

Benachi, A., A. Yamgnane. M. Olivi, Y. Dumez, E. Gautier, J. M. Costa (2005) Clin Chem 51:242-244.
Casilli, F., Z. C. Di Rocco, et al. (2002). “Rapid detection of novel BRCA1 rearrangements in high-risk breast-ovarian cancer families using multiplex PCR of short fluorescent fragments.” Hum Mutat 20(3): 218-26.
Chinnapapagari, S. K., W. Holzgreve, et al. (2005). “Treatment of maternal blood samples with formaldehyde does not alter the proportion of circulatory fetal nucleic acids (DNA and mRNA) in maternal plasma.” Clin Chem 51(3): 652-5.
Chiu, R. W. K., T. K. Lau, T. N. Leung, K. C. K. Chow, D. H. K. Chui, Y. M. D. Lo (2002) Lancet 360:998-1000
Chung, G. T. Y, R. W. K. Chiu, K. C. A Chan, T. K. Lau, T. N. Leung, Y. M. D., Lo (2005) Clin Chem 51:655-658.
Costa, J. M., A. Benachi, E. Gautier (2002) N Engl J Med 346:1502.
Dhallan R, Guo X, Emche S, Damewood M, Bayliss P, Cronin M, Barry J, Betz J, Franz K, Gold K, et al. (2007) Lancet 369:474-481.
Fan, H. C. and S. R. Quake (2007). “Detection of aneuploidy with digital polymerase chain reaction.” Anal Chem 79(19): 7576-9.
Lee, T., E. S. LeShane, et al. (2002). “Down syndrome and cell-free fetal DNA in archived maternal serum.” Am J Obstet Gynecol 187(5): 1217-21.
Li, Y., B. Zimmermann, et al. (2004). “Size separation of circulatory DNA in maternal plasma permits ready detection of fetal DNA polymorphisms.” Clin Chem 50(6): 1002-11.
Lo Y. M. D., R. W. K. Chiu R W K (2007) Nat Rev Genet 8:71-77.
Lo, Y. M. and R. W. Chiu (2008). “Noninvasive prenatal diagnosis of fetal chromosomal aneuploidies by maternal plasma nucleic acid analysis.” Clin Chem 54(3): 461-6.
Lo, Y. M., N. Corbetta, et al. (1997). “Presence of fetal DNA in maternal plasma and serum.” Lancet 350(9076): 485-7.
Lo, Y. M. D., N. M. Hjelm, C. Fidler, I. L. Sargent, M. F. Murphy, P F. Chamberlain, P. M. Poon, C. W. Redman, J. S. Wainscoat (1998) N Engl J Med 339:1734-1738.
Lo, Y. M., F. M. Lun, et al. (2007). “Digital PCR for the molecular detection of fetal chromosomal aneuploidy.” Proc Natl Acad Sci USA 104(32): 13116-21.
Lo, Y. M., M. S. Tein, et al. (1998). “Quantitative analysis of fetal DNA in maternal plasma and serum: implications for noninvasive prenatal diagnosis.” Am J Hum Genet 62(4): 768-75.
Spencer, K. (2007). “Aneuploidy screening in the first trimester.” Am J Med Genet C Semin Med Genet 145C(1): 18-32.
Thorsen, T., S. J. Maerkl, et al. (2002). “Microfluidic large-scale integration.” Science 298(5593): 580-4.
Wachtel, S. S., L. P. Shulman, et al. (2001). “Fetal cells in maternal blood.” Clin Genet 59(2): 74-9.
Zimmermann, B. G., S. Grill, W. Holzgreve, X. Yan Zhong, L. G. Jackson, S. Hahn (2008) “Digital PCR: a powerful new tool for noninvasive prenatal diagnosis?” Prenat Diagn 28: 1087-1093.

1. A method of detecting relative target source to reference source ratios in a biological sample, the method comprising:
(a) distributing the biological sample into discrete subsamples, wherein the biological sample comprises
a plurality of target molecules corresponding to spaced apart target sites on a target source; and
a plurality of reference molecules corresponding to spaced apart reference sites on a reference source;
(b) providing a target primer subset of at least one digital amplification target primer pair directed to one or more of the plurality of target molecules and a reference primer subset of at least one digital amplification reference primer pair directed to one or more of the plurality of reference molecules;
(c) performing digital amplification with the target primer subset and the reference primer subset; and
(d) detecting the presence or absence of amplified target products with a subset of target probes and detecting the presence or absence of amplified reference products with a subset of reference probes, wherein the number of target probes and the number reference probes is less than the number of target and reference sites respectively, and wherein the ratio of amplified target products to amplified reference products is indicative of a relative amount of target source to reference source in a biological sample.

2. The method of claim 1, wherein the target probes identify a common template-derived internal probe target sequence and the reference probes identify a common template-derived internal probe reference sequence.

3. The method of claim 1, wherein the target primer subset primer pairs are combination probe and primer molecules and the reference primer subset primer pairs are combination probe and primer molecules.

4. The method of claim 1, further comprising, a pre-amplification prior to distributing the biological sample into discrete subsamples, wherein the pre-amplification comprises:
(a) providing a subset of target pre-amplification primer pairs, wherein each target pre-amplification primer pair is specific to at least one target site, and a subset of reference pre-amplification primer pairs, wherein each reference pre-amplification primer pair is specific to at least one reference site, and wherein each target pre-amplification primer pair has a first universal non-specific primer sequence and wherein each reference pre-amplification primer pair has a second universal non-specific primer sequence; and
(b) performing a pre-amplification reaction on the biological sample with the first and second subsets to synthesize the target molecules and the reference molecules.

5. The method of claim 1, wherein the ratio of amplified target products to amplified reference products is indicative of the presence or absence of a chromosomal abnormality.

6. The method of claim 1, wherein the target source is all or part of a chromosome and the reference source is all or part of a different chromosome.

7. The method of claim 1, wherein the target source is selected from human chromosomes: X; Y; 8; 9; 12; 13; 16; 18; 21; and 22.

8. The method of claim 1, wherein the reference source is selected from human chromosomes: 1; 2; 3; 4; 5; 6; 7; 10; 11; 14; 15; 17; 19; and 20.

9. The method of claim 1, wherein the target primer subset of primers are added to a first subset of discrete subsamples and the reference primer subset of primers are added to a second subset of discrete subsamples.

10. The method of claim 1, wherein the discrete subsamples comprise on average about 1.59 target molecules or reference molecules.

11. The method of claim 1, wherein the discrete subsamples have between 0-3 target or reference molecules.

12. The method of claim 1, wherein the target primer subset is directed to at least 8 target sites and the reference primer subset is directed to at least 8 reference sites.

13. The method of claim 1, wherein each site on the target source is separated by at least 10 kb and each site on the reference source is separated by at least 10 kb.

14. The method of claim 1, wherein the biological sample is blood, blood plasma, blood serum, urine, feces, saliva, or transcervical lavage.

15. The method of claim 1, wherein the biological sample is maternal blood serum.

16. The method of claim 1, wherein the ratio of amplified target products to amplified reference products is indicative of the presence or absence of fetal aneuploidy.

17. The method of claim 1, wherein the target source is human chromosome 21.

18. The method of claim 1, wherein the ratio of amplified target products to amplified reference products is indicative of the presence or absence of trisomy 21 in the fetal chromosomes.

19. The method of claim 1, wherein the at least one digital amplification target primer pair is selected from one or more of the following primer pairs:
(a) SEQ ID NO: 1 and SEQ ID NO: 2;
(b) SEQ ID NO: 3 and SEQ ID NO: 4;
(c) SEQ ID NO: 5 and SEQ ID NO: 6;
(d) SEQ ID NO: 7 and SEQ ID NO: 8;
(e) SEQ ID NO: 9 and SEQ ID NO: 10;
(f) SEQ ID NO: 11 and SEQ ID NO: 12;
(g) SEQ ID NO: 13 and SEQ ID NO: 14; and
(h) SEQ ID NO: 15 and SEQ ID NO: 16.

20. A method of detecting a relative amount of target source to reference source in a biological sample, the method comprising:
(a) distributing the biological sample into discrete subsamples, wherein the biological sample comprises
a plurality of target molecules corresponding to spaced apart target sites on a target source; and
a plurality of reference molecules corresponding to spaced apart reference sites on a reference source;
(b) providing a target primer subset of at least one digital amplification target primer pair directed to one or more of the plurality of target molecules and a reference primer subset of at least one digital amplification reference primer pair directed to one or more of the plurality of reference molecules;
(c) performing digital amplification with the target primer subset and the reference primer subset; and
(d) detecting the presence or absence of amplified target products with a subset of target probes and detecting the presence or absence of amplified reference products with a subset of reference probes, wherein the target probes have a common template-derived internal probe target sequence and the reference probes have a common template-derived internal probe reference sequence, and wherein the ratio of amplified target products to amplified reference products is indicative of a relative amount of target source to reference source in a biological sample.

21. The method of claim 20, wherein the target primer subset of primers are added to a first subset of discrete subsamples and the reference primer subset of primers are added to a second subset of discrete subsamples.

22. The method of claim 20, wherein the discrete subsamples comprise on average about 1.59 target molecules or reference molecules.

23. The method of claim 20, wherein the discrete subsamples have between 0-3 target or reference molecules.

24. The method of claim 20, wherein the target primer subset is directed to at least 8 target sites and the reference primer subset is directed to at least 8 reference sites.

25. The method of claim 20, wherein each site on the target source is separated by at least 10 kb and each site on the reference source is separated by at least 10 kb.

26. The method of claim 20, wherein the biological sample is blood, blood plasma, blood serum, urine, saliva, or transcervical lavage.

27. The method of claim 20, wherein the biological sample is maternal blood serum.

28. The method of claim 20, wherein ratio of amplified target products to amplified reference products is indicative of the presence or absence of fetal aneuploidy.

29. The method of claim 20, wherein the target source is human chromosome 21.

30. The method of claim 20, wherein ratio of amplified target products to amplified reference products is indicative of the presence or absence of trisomy 21 in the fetal chromosomes.

31. The method of claim 20, wherein the at least one digital amplification target primer pair is selected from one or more of the following primer pairs:
(a) SEQ ID NO: 1 and SEQ ID NO: 2;
(b) SEQ ID NO: 3 and SEQ ID NO: 4;
(c) SEQ ID NO: 5 and SEQ ID NO: 6;
(d) SEQ ID NO: 7 and SEQ ID NO: 8;
(e) SEQ ID NO: 9 and SEQ ID NO: 10;
(f) SEQ ID NO: 11 and SEQ ID NO: 12;
(g) SEQ ID NO: 13 and SEQ ID NO: 14; and
(h) SEQ ID NO: 15 and SEQ ID NO: 16.

32. A method of detecting the relative amount of target source to reference source in a biological sample, the method comprising:
(a) providing a subset of target pre-amplification primer pairs, wherein each target pre-amplification primer pair is specific to at least one target site, and a subset of reference pre-amplification primer pairs, wherein each reference pre-amplification primer pair is specific to at least one reference site, and wherein each target pre-amplification primer pair has a first universal non-specific primer sequence and wherein each reference pre-amplification primer pair has a second universal non-specific primer sequence; and
(b) performing a pre-amplification reaction on the biological sample with the first and second subsets to synthesize the target molecules and the reference molecules, wherein the biological sample comprises additional target and reference molecules;
(c) distributing the biological sample into discrete subsamples, wherein the biological sample comprises
a plurality of target molecules corresponding to spaced apart target sites on a target source; and
a plurality of reference molecules corresponding to spaced apart reference sites on a reference source;
(d) providing a target primer subset of at least one digital amplification target primer pair directed to one or more of the plurality of target molecules and a reference primer subset of at least one digital amplification reference primer pair directed to one or more of the plurality of reference molecules;
(e) performing digital amplification with the target primer subset and the reference primer subset; and
(f) detecting the presence or absence of amplified target products with a subset of target probes and detecting the presence or absence of amplified reference products with a subset of reference probes, wherein the number of target probes and the number reference probes is less than the number of target and reference sites respectively, and wherein the ratio of amplified target products to amplified reference products is indicative of a relative amount of target source to reference source in a biological sample.

33. The method of claim 32, wherein the target primer subset of primers are added to a first subset of discrete subsamples and the reference primer subset of primers are added to a second subset of discrete subsamples.

34. The method of claim 32, wherein the discrete subsamples comprise on average about 1.59 target molecules or reference molecules.

35. The method of claim 32, wherein the discrete subsamples have between 0-3 target or reference molecules.

36. The method of claim 32, wherein the target primer subset is directed to at least 8 target sites and the reference primer subset is directed to at least 8 reference sites.

37. The method of claim 32, wherein each site on the target source is separated by at least 10 kb and each site on the reference source is separated by at least 10 kb.

38. The method of claim 32, wherein the biological sample is blood, blood plasma, blood serum, urine, saliva, or transcervical lavage.

39. The method of claim 32, wherein the biological sample is maternal blood serum.

40. The method of claim 32, wherein ratio of amplified target products to amplified reference products is indicative of the presence or absence of fetal aneuploidy.

41. The method of claim 32, wherein the target source is human chromosome 21.

42. The method of claim 32, wherein ratio of amplified target products to amplified reference products is indicative of the presence or absence of trisomy 21 in the fetal chromosomes.

43. A kit for detecting the relative amount of target source to reference source in a biological sample, the kit comprising:
(a) a target primer subset of at least 8 digital amplification target primer pairs directed to a plurality of target molecules and a reference primer subset of at least 8 digital amplification reference primer pairs directed to a plurality of reference molecules, wherein the target molecules are situated on a human chromosome selected from the following: X; Y; 8; 9; 12; 13; 16; 18; 21; and 22 and wherein the reference molecules are situated on a human chromosome selected from the following: 1; 2; 3; 4; 5; 6; 7; 10; 11; 14; 15; 17; 19; and 20;
(b) an amplification buffer for digital amplification using the target primer pairs and the reference primer pairs; and
(c) a target probe specific for the target primer pair amplification products and a reference probe specific for the reference primer pair amplification products.

44. The kit of claim 43, wherein the at least one digital amplification target primer pair is selected from one or more of the following primer pairs:
(a) SEQ ID NO: 1 and SEQ ID NO: 2;
(b) SEQ ID NO: 3 and SEQ ID NO: 4;
(c) SEQ ID NO: 5 and SEQ ID NO: 6;
(d) SEQ ID NO: 7 and SEQ ID NO: 8;
(e) SEQ ID NO: 9 and SEQ ID NO: 10;
(f) SEQ ID NO: 11 and SEQ ID NO: 12;
(g) SEQ ID NO: 13 and SEQ ID NO: 14; and
(h) SEQ ID NO: 15 and SEQ ID NO: 16.


SEQUENCE LISTING (16 sequences)

SEQ ID NO: 1 - DNA, 40 bp, Artificial Sequence
  a universal primer target sequence and a human Chromosome 21 sequence-specific sequence
gttgtaaaacgacggccagtacggaagcagaggcttctaa

SEQ ID NO: 2 - DNA, 40 bp, Artificial Sequence
  a universal primer target sequence and a human Chromosome 21 sequence-specific sequence
cacaggaaacagctatgacctccgagtgttttcagatgga

SEQ ID NO: 3 - DNA, 38 bp, Artificial Sequence
  a universal primer target sequence and a human Chromosome 21 sequence-specific sequence
gttgtaaaacgacggccagtagcaggtggggtggattt

SEQ ID NO: 4 - DNA, 39 bp, Artificial Sequence
  a universal primer target sequence and a human Chromosome 21 sequence-specific sequence
cacaggaaacagctatgaccaacctgggaggcagcttag

SEQ ID NO: 5 - DNA, 40 bp, Artificial Sequence
  a universal primer target sequence and a human Chromosome 21 sequence-specific sequence
gttgtaaaacgacggccagtcatgtcagccgagtctcctc

SEQ ID NO: 6 - DNA, 38 bp, Artificial Sequence
  a universal primer target sequence and a human Chromosome 21 sequence-specific sequence
cacaggaaacagctatgacccctggcaattgtccacct

SEQ ID NO: 7 - DNA, 39 bp, Artificial Sequence
  a universal primer target sequence and a human Chromosome 21 sequence-specific sequence
gttgtaaaacgacggccagtgtcttcacgagcgtgcatc

SEQ ID NO: 8 - DNA, 41 bp, Artificial Sequence
  a universal primer target sequence and a human Chromosome 21 sequence-specific sequence
cacaggaaacagctatgaccaaacccgtgtctgactcactg

SEQ ID NO: 9 - DNA, 42 bp, Artificial Sequence
  a universal primer target sequence and a human Chromosome 21 sequence-specific sequence
gttgtaaaacgacggccagtgccttacatgtgaaagtgcgta

SEQ ID NO: 10 - DNA, 41 bp, Artificial Sequence
  a universal primer target sequence and a human Chromosome 21 sequence-specific sequence
cacaggaaacagctatgaccgctctctgaatcctgtgtggt

SEQ ID NO: 11 - DNA, 40 bp, Artificial Sequence
  a universal primer target sequence and a human Chromosome 21 sequence-specific sequence
gttgtaaaacgacggccagtgggcactgactggtctaagg

SEQ ID NO: 12 - DNA, 42 bp, Artificial Sequence
  a universal primer target sequence and a human Chromosome 21 sequence-specific sequence
cacaggaaacagctatgacccacaactccagactggctacag

SEQ ID NO: 13 - DNA, 41 bp, Artificial Sequence
  a universal primer target sequence and a human Chromosome 21 sequence-specific sequence
gttgtaaaacgacggccagtgagaccacgctagtcacagga

SEQ ID NO: 14 - DNA, 40 bp, Artificial Sequence
  a universal primer target sequence and a human Chromosome 21 sequence-specific sequence
cacaggaaacagctatgaccggtgagaagaggcagtgagc

SEQ ID NO: 15 - DNA, 42 bp, Artificial Sequence
  a universal primer target sequence and a human Chromosome 21 sequence-specific sequence
gttgtaaaacgacggccagtcccagggacacagctagtaagt

SEQ ID NO: 16 - DNA, 38 bp, Artificial Sequence
  a universal primer target sequence and a human Chromosome 21 sequence-specific sequence
cacaggaaacagctatgaccagattctgccgccatcct